(19) United States
(12) Patent Application Publication
(10) Pub. No.: US 2026/0021159 A1
(43) Pub. Date: Jan. 22, 2026

(54) STABLE PEPTIDE COMPOSITIONS AND METHODS OF USE THEREOF FOR TREATMENT OF MODERATE/SEVERE DRY EYE-RELATED OCULAR SYMPTOMS

(71) Applicant: TearSolutions, Inc., Charlottesville, VA (US)

(72) Inventors: Anil Asrani, Plymouth, MN (US); Gordon W. Laurie, Charlottesville, VA (US); Marc G. Odrich, Crozet, VA (US)

(21) Appl. No.: 18/880,209

(22) PCT Filed: Jun. 21, 2023
(86) PCT No.: PCT/US2023/068835

§ 371 Date: Dec. 30, 2024

Related U.S. Application Data
(60) Provisional application No. 63/357,638, filed on Jul. 1, 2022.
(60) Provisional application No. 63/390,550, filed on Jul. 19, 2022.

(51) Int. Cl. A61K 38/10; A61K 9/00; A61K 47/02; A61K 47/12; A61K 47/32; A61P 27/02

(52) U.S. Cl. CPC A61K 38/10; A61K 9/0048; A61K 47/02; A61K 47/12; A61K 47/32; A61P 27/02

(57) ABSTRACT

This application generally relates to stable peptide compositions and kits comprising low levels of buffering and chelating agents, and methods of using the same for the treatment of patients having moderate/sever or severe dry eye.

### CROSS-REFERENCE TO RELATED APPLICATIONS

This application claims priority to U.S. Ser. No. 63/357,638, filed Jul. 1, 2022, and to U.S. Ser. No. 63/390,550, filed Jul. 19, 2023, each of which is herein incorporated by reference in its entirety.

### BACKGROUND

### Field

The present application relates to the fields of chemistry, biochemistry and medicine. More particularly, several embodiments of the present application relate to stable, dilute peptide compositions and kits comprising such compositions, for use in the treatment of ocular disorders, in particular, moderate/severe or severe dry eye.

### Description of the Related Art

While dry eye treatments are commercially available, there remains a need for an effective treatment for general dry eye, particularly in patients with moderate/severe or severe dry eye symptoms.

### SUMMARY

There is an unmet need for effective treatment of dry eye, particularly in patients with symptoms of moderate/severe dry eye as disclosed herein (e.g., those with Eye Dryness Scores of at least 60). Further, there is an unmet need for peptide compositions that provide therapeutic amounts of peptides, are stable at room temperature, and contain only trace amounts of stabilizers and/or preservatives, or none at all.

Non-limiting embodiments of the present disclosure include the following numbered embodiments:
1. A method of treating a moderate/severe dry eye-related ocular symptom, comprising:
   identifying a subject suffering from moderate/severe dry eye-related ocular symptoms; and
   administering to the subject a liquid composition comprising:
   0.0001-0.005% (w/v) of a polypeptide or a pharmaceutically acceptable salt thereof, the polypeptide having a sequence consisting of Ac-Lys-Gln-Phe-Ile-Glu-Asn-Gly-Ser-Glu-Phe-Ala-Gln-Lys-Leu-Leu-Lys-Lys-Phe-Ser-NH2 (SEQ ID NO: 1), wherein “Ac” represents an acetyl group and the C-terminus is amidated;
   0.01-0.6% (w/v) of a buffer;
   0.0005-0.01% (w/v) disodium EDTA;
   0.0001-0.01% (w/v) or 0.0001-0.05% (w/v) tyloxapol;
   and 0.1-1.0% (w/v) sodium chloride,
   wherein the pH of the composition is about 6.2 to about 6.8,
   wherein one drop of the composition is administered to an eye of the subject up to three times daily.
2. The method of embodiment 1, wherein the subject satisfies at least one of the criteria selected from the group consisting of:
   a) FCS total score ≥4 and <15 in the NET/Industry Workshop scale;
   b) Eye Dryness score of ≥60 using the VAS instantaneous measure;
   c) Anesthetized Schirmer 1 test score <5 mm wetting/5 min; and
   d) LGCS total score >5 using the NEI/Industry Workshop scale (where 0=no staining).
3. The method of embodiment 1 or 2, wherein the subject has an Eye Dryness score of ≥60 using the VAS instantaneous measure.
4. The method of embodiment 1 or 2, wherein the subject satisfies all 4 criteria in a single eye.
5. The method of any one of embodiments 1-4 wherein the subject does not have Sjögren's Syndrome.
6. The method of any one of the preceding embodiments, wherein the polypeptide or pharmaceutically acceptable salt thereof is present in the liquid composition at 0.00025-0.005% (w/v), or at 0.0001-0.001% (w/v).
7. The method of any one of the preceding embodiments, wherein the polypeptide or pharmaceutically acceptable salt thereof is present in the liquid composition at 0.00025% or 0.001% (w/v).
8. The method of any one of the preceding embodiments, wherein the tyloxapol is present in the liquid composition at 0.0005-0.01% (w/v) or 0.0005-0.05% (w/v).
9. The method of any one of the preceding embodiments, wherein the tyloxapol is present in the liquid composition at about 0.001% (w/v) or about 0.01% (w/v) or about 0.05% (w/v).
10. The method of any one of the preceding embodiments, wherein the pH of the composition is about 6.0 to about 7.0, or about 6.4 to about 6.6.
11. The method of embodiment 9 or 10, wherein the pH of the composition is about 6.5.
12. The method of any one of the preceding embodiments, wherein the amount of NaCl is about 0.4% to about 0.6% (w/v).
13. The method of any one of the preceding embodiments, wherein the amount of NaCl is about 0.5% (w/v).
14. The method of embodiment 11 or 13, wherein the osmolality of the composition is about 190 to 210 mOsm/kg.
15. The method of any one of the preceding embodiments, wherein the buffer is a citrate buffer.
16. The method of embodiment 13 or 15, wherein the citrate buffer comprises 0.0098% anhydrous citric acid and 0.279% sodium citrate dihydrate.
17. The method of any one of the preceding embodiments, wherein the amount of EDTA is about 0.0005% to about 0.005% (w/v).
18. The method of any one of the preceding embodiments, wherein the amount of EDTA is about 0.001% (w/v).
19. The method of any of any one of the preceding embodiments, wherein the composition further comprises 0.04% methylparaben.
20. The method of any of any one of the preceding embodiments, wherein the composition is sterile.
21. The method of any one of the preceding embodiments, further comprising determining that the subject has a history of one or more dry eye-related ocular symptoms.
22. The method of any one of the preceding embodiments, wherein the one or more dry eye-related ocular symptoms comprises the subject's use of ocular wetting agents within the last 6 months.
23. The method of any one of the preceding embodiments, wherein the composition is administered three times daily.
24. The method of any one of the preceding embodiments, wherein the composition is administered up to three times daily for at least one week.
25. The method of any one of the preceding embodiments, wherein the composition is administered up to three times daily for 1-6 weeks.
26. The method of any one of the preceding embodiments, wherein the administration improves the FCS total score (NEI/Industry Workshop 0-15 scale) in the subject's eye after at least two weeks of treatment, or after at least four weeks of treatment, or after at least six weeks from the start of four weeks of treatment, compared to a baseline measure prior to starting treatment.
27. The method of any one of the preceding embodiments, wherein the administration improves one or more of:
   eye dryness after at least two weeks of treatment, or after at least four weeks of treatment, compared to baseline on a visual analog scale,
   SANDE (global scores SANDE 1) after at least two weeks of treatment compared to a baseline measure prior to starting treatment;
   Mean Scores for SANDE (global scores SANDE-1) after at least two weeks of treatment compared to a baseline measure prior to starting treatment,
   Individual Symptom Assessments (Instantaneous) after at least two weeks of treatment compared to a baseline measure prior to starting treatment,
   Mean Scores for Individual Symptom Assessments (Reflective) after at least two weeks of treatment compared to a baseline measure prior to starting treatment;
   LGCS in the subject's eye after at least two weeks of treatment compared to a baseline measure prior to starting treatment;
   Anesthetized Schirmer test in the subject's eye after at least two weeks of treatment compared to a baseline measure prior to starting treatment;
   TFBUT in the subject's eye after at least two weeks of treatment compared to a baseline measure prior to starting treatment;
   FCS in the subject's eye after at least two weeks of treatment compared to a baseline measure prior to starting treatment;
   SANDE (global scores for SANDE 1) after at least 2 weeks of treatment, or after at least 4 weeks of treatment, or 1 week after 4 weeks treatment compared to a baseline measure prior to starting treatment;
   Individual Symptoms (Instantaneous) after at least 2 weeks of treatment, or after at least 4 weeks of treatment, or 1 week after 4 weeks treatment compared to a baseline measure prior to starting treatment;
   Mean Scores for (global scores SANDE-2) after at least 2 weeks of treatment, or after at least 4 weeks of treatment, or 1 week after 4 weeks treatment compared to a baseline measure prior to starting treatment;
   Mean Scores for Individual Symptom Assessments (Reflective) after at least 2 weeks of treatment, or after at least 4 weeks of treatment, or 1 week after 4 weeks treatment compared to a baseline measure prior to starting treatment;
   FCS and SANDE 1 and Individual Symptom Assessments (Instantaneous) after at least 2 weeks of treatment, or after at least 4 weeks of treatment, compared to a baseline measure prior to starting treatment;
   LGCS after at least 2 weeks of treatment, or after at least 4 weeks of treatment compared to a baseline measure prior to starting treatment,
   Anesthetized Schirmer test results after at least 2 weeks of treatment, or after at least 4 weeks of treatment, compared to a baseline measure prior to starting treatment;
   TFBUT after at least 2 weeks of treatment, or after at least 4 weeks of treatment, or 1 week after 4 weeks treatment compared to a baseline measure prior to starting treatment.
28. The method of embodiment 27, the improvement is, is about, is at least, is at least about, 5, 10, 15, 20, 25, 30, 35, 40, 45, 50, 55, 60, 65, 70, 75, 80, 85, 90, 95, or 100% of the value of the measure or assessment, or a range defined by any two of the preceding values.
29. The method of any one of the preceding embodiments, wherein the subject further meets all the following criteria:
   18 years of age or older;
   a documented prior history or current diagnosis of dry eye-related ocular symptoms; and
   a history of dry eye-related ocular symptoms, and who has self-reported use of over the counter ocular wetting agents within the last 120 days.
30. The method of any one of the preceding embodiments, wherein the subject does not meet one or more of the following criteria:
   any active infectious ocular condition;
   monocular or have a Best Corrected Visual Acuity (BCVA), using corrective lenses if necessary, of +1.0 logMAR or worse as assessed by Early Treatment Diabetic Retinopathy Study (ETDRS);
   ocular inflammatory conditions (e.g., conjunctivitis, keratitis, anterior blepharitis, etc.) not related to dry eye syndrome;
   clinical evidence of cicatricial ocular surface disease, such as cicatricial ocular pemphigoid or Stevens Johnson syndrome;
   cannot suspend the use of any topical eye medications (including topical cyclosporine) other than the investigational product during the run-in and the treatment phase;
   has used Restasis® (topical ophthalmic cyclosporine) within 60 days prior to beginning treatment with the composition;
   has used Xiidra® (topical ophthalmic lifitegrast) within 60 days prior to beginning treatment with the composition;
   the subject's eye has fluorescein corneal staining (FCS) total score=15 or a score=3 in the superior region NEI/Industry Workshop scale or the subject's eye has FCS with diffuse confluent staining, filaments or epithelial defects;
   has active or have had an outbreak of herpetic keratitis within 365 days of beginning treatment or subjects who are on chronic oral antivirals for herpetic disease;
   cannot suspend the use of and abstain from contact lens use during treatment;
   has used or anticipate use of amiodarone;
   within 30 days prior to beginning treatment alter the dose or anticipate alterations to the dose of the following: tetracyclines, Omega 3 or Omega 6;
   who within 60 days prior to beginning treatment and/or for the duration of treatment, altered the dose or anticipate alterations to the dose of the following: anticholinergics, antidepressants, oral contraceptives, isotretinoin, oral systemic corticosteroids, oral systemic immunosuppressive agents,
   within 30 days prior to beginning treatment and/or for the duration of the treatment have used topical ocular antihistamines, ocular, inhaled or intranasal corticosteroids, topical or oral mast cell stabilizers, oral antihistamines, topical or nasal vasoconstrictors, topical ocular NSAIDs, topical ocular antibiotics;
   in the subject's eye and within the past 90 days have had cauterization of the punctum or alterations to (insertion or removal) punctal plug(s) before beginning treatment;
   in the subject's eye, have had corneal refractive surgery (LASIK, PRK, RK);
   has a history of any operative procedure on the ocular surface or eyelids within 365 days prior to beginning treatment with a history of intraocular surgery within 90 days prior to beginning treatment;
   is pregnant or suspected to be pregnant;
   is breastfeeding or intend to breastfeed;
   has any physical or mental impairment that would preclude participation and the ability to give informed consent; and
   has participated in a device or investigational drug study or clinical trial within 30 days of beginning treatment.
31. The method of any one of the preceding embodiments, wherein the subject does not meet at least one of the following criteria in one eye, or in both eyes:
   a) FCS total score <4 or >15 in the NEI/Industry Workshop scale,
   b) Eye Dryness score of <60 using the VAS instantaneous,
   c) Anesthetized Schirmer 1 test score >5 mm wetting/5 min, or
   d) LGCS total score <5 using the NEI/Industry Workshop scale (where 0=no staining).
32. The method of any one of the preceding embodiments, wherein the subject is identified as suffering from severe dry eye, rather than moderate/severe dry eye.
33. The method of embodiment 32, wherein the subject has Eye Dryness score of ≥70 using the VAS instantaneous measure in at least one eye.
34. The method of any one of the preceding embodiments, wherein the composition comprises, consists, or consists essentially of, a composition selected from the compositions of a formula in Tables A, B, C, 1.1, 1.2 and 1.3.
35. A liquid composition for use in treating a moderate/severe dry eye-related ocular symptom in an eye of a subject, the composition comprising:
   0.0001-0.006% (w/v) of a polypeptide or a pharmaceutically acceptable salt thereof, the polypeptide having a sequence consisting of Ac-Lys-Gln-Phe-Ile-Glu-Asn-Gly-Ser-Glu-Phe-Ala-Gln-Lys-Leu-Leu-Lys-Lys-Phe-Ser-NH2 (SEQ ID NO: 1), wherein “Ac” represents an acetyl group and the C-terminus is amidated;
   0.01-0.6% (w/v) of a buffer;
   0.0005-0.01% (w/v) disodium EDTA;
   0.0001-0.01% (w/v) or 0.0001-0.05% (w/v) tyloxapol;
   and 0.1-1.0% (w/v) sodium chloride,
   wherein the pH of the composition is about 6.2 to about 6.8.
36. The liquid composition of embodiment 35, wherein the composition comprises, consists, or consists essentially of, a composition selected from the compositions of a formula in Tables A, B, C, 1.1, 1.2 and 1.3.
37. The liquid composition of any one of embodiments 35-36, wherein the subject satisfies at least one of the criteria selected from the group consisting of:
   a) FCS total score >4 and <15 in the NEI/Industry Workshop scale;
   b) Eye Dryness score of ≥60 using the VAS instantaneous measure;
   c) Anesthetized Schirmer 1 test score ≤5 mm wetting/5 min; and
   d) LGCS total score >5 using the NEI/Industry Workshop scale (where 0=no staining).
38. The liquid composition of any one of embodiments 35-37, wherein the subject has an Eye Dryness score of ≥60 using the VAS instantaneous measure.
39. The liquid composition of any one of embodiments 35-38, wherein the subject satisfies all 4 criteria in a single eye.
40. The liquid composition of any one of embodiments 35-39, wherein the subject does not have Sjögren's Syndrome.
41. The liquid composition of any one of embodiments 35-40, wherein the subject does not meet at least one at least one of the following criteria in one eye, or in both eyes:
   a) FCS total score <4 or ≥15 in the NEI/Industry Workshop scale,
   b) Eye Dryness score of <60 using the VAS instantaneous,
   c) Anesthetized Schirmer 1 test score >5 mm wetting/5 min, or
   d) LGCS total score <5 using the NEI/Industry Workshop scale (where 0=no staining).
42. The liquid composition of any one of embodiments 35-41, wherein the subject is identified as suffering from severe dry eye, rather than moderate/severe dry eye.
43. The liquid composition of embodiment 42, wherein the subject has Eye Dryness score of ≥70 using the VAS instantaneous measure in at least one eye.

### BRIEF DESCRIPTION OF THE DRAWINGS

FIG. 1 is a schematic diagram depicting an embodiment of a method of treating a subject with moderate/severe dry eye symptoms, according to some non-limiting embodiments of the present disclosure.

FIG. 2 is a schematic diagram showing the study design for treatment of Primary Sjögren's associated ocular surface disease with Lacripep™.

FIG. 3 is a graph showing change in inferior CFS from baseline after 3 times daily ocular administration of Lacripep™ at 22 μM at 14 and 28 days, in Primary Sjögren's Syndrome patients.

FIG. 4A is a graph showing change in inferior CFS from baseline after 3 times daily ocular administration of placebo, 22 μM or 44 μM Lacripep™ at 14 or 28 days, and at post-treatment follow-up (Day 42), in Primary Sjögren's Syndrome patients.

FIG. 4B is a graph showing change in inferior CFS from baseline after 3 times daily ocular administration of placebo, 22 μM or 44 μM Lacripep™ at 14 or 28 days, and at post-treatment follow-up (Day 42), in a subgroup of Primary Sjögren's Syndrome patients having EDS >60.

FIG. 5A is a graph showing change in burning/stinging symptom from baseline after 3 times daily ocular administration of placebo, 22 μM or 44 μM Lacripep™ at 14 or 28 days, and at post-treatment follow-up (Day 42), in Primary Sjögren's Syndrome patients.

FIG. 5B is a graph showing change in burning/stinging symptom from baseline after 3 times daily ocular administration of placebo, 22 μM or 44 μM Lacripep™ at 14 or 28 days, and at post-treatment follow-up (Day 42), in a subgroup of Primary Sjögren's Syndrome patients having EDS >60.

FIG. 6 is an embodiment of a VAS questionnaire for measuring an Eye Dryness Score (EDS).

FIG. 7 is an embodiment of a schematic diagram showing the division of the corneal surface for measuring the fluorescein corneal staining (FCS) total score.

FIG. 8 is an embodiment of a schematic diagram showing the division of the conjunctival surface for measuring the Lissamine green conjunctival staining (LGCS) total score.

### DETAILED DESCRIPTION

### Terms

The following are illustrative definitions of terms used herein. Unless expressly stated otherwise, all technical and scientific terms used herein have the same meaning as is commonly understood by one of ordinary skill in the art read in light of the entire specification. All patents, applications, published applications and other publications referenced herein are incorporated by reference in their entirety unless stated otherwise.

The term “about,” as used herein, refers to a quantity, value, number, frequency, percentage, amount, or weight that varies +/−10% to a reference quantity, value, number, frequency, percentage, amount, or weight.

Unless indicated otherwise, when a percentage (%) value is used in the present application, the value refers to a weight/volume (w/v) percent value.

The term “tonicity agent” as used herein, shall be given its ordinary meaning and shall include materials whose primary purpose is to alter the osmolality of a composition. Suitable tonicity agents include, but are not limited to, propylene glycol, polyethylene glycols, sodium chloride, potassium chloride, magnesium chloride, calcium chloride, simple sugars such as dextrose, fructose, galactose, and/or simple polyols such as the sugar alcohols mannitol, sorbitol, xylitol, lactitol, isomaltitol, maltitol, hydrogenated starch hydrolysates, glycerin, and combinations of the foregoing.

The term “stabilizing agent” as used herein shall be given its ordinary meaning and shall include a material that inhibits chemical reactions with a peptide. Stabilizing agents may include, for example, antioxidants such as sodium metabisulfite, sodium thiosulfate, acetylcysteine, butylated hydroxyanisole, butylated hydroxytoluene, and combinations of the foregoing.

The term “surfactant” as used herein shall be given its ordinary meaning and shall include amphiphilic molecules, meaning that they contain both hydrophobic groups (tails) and hydrophilic groups (heads). Therefore, a surfactant contains both a water insoluble (or oil soluble) component and a water soluble component. As used herein, surfactants may be detergents, wetting agents, emulsifiers, foaming agents, or dispersants. In some embodiments, the polypeptide can act as a surfactant.

The term “chelating agent,” as used herein shall be given its ordinary meaning and shall include a compound that can form two or more bonds to a metal ion, i.e., a multi-dentate ligand. Chelating agents include, but are not limited to ethylenediaminetetraacetic acid (EDTA), ethylenediamine, amino acids such as glutamic acid and histidine, organic diacids such as oxalic acid, malonic acid, succinic acid, and the like, and pharmaceutically acceptable salts of the foregoing. In several embodiments, a chelating agent is EDTA, or a pharmaceutically acceptable salt thereof. In some embodiments, the polypeptide can act as a chelator.

The term “viscosity building agent” as used herein, shall be given its ordinary meaning and shall include materials that affect the viscosity (centipoise, or Cp) of a composition. Examples of viscosity enhancing agents include, but are not limited to: polysaccharides, such as hyaluronic acid and its salts, chondroitin sulfate and its salts, dextrans, various polymers of the cellulose family (and derivatives thereof), vinyl polymers, and acrylic acid polymers. Non-limiting examples of viscosity building agents include polyvinylalcohol (PVA), polyvinylpyrrolidone (PVP), polyethylene glycol (PEG), and polyacrylic acid (PAA).

The term “ophthalmically acceptable” as used herein shall be given its ordinary meaning and shall include materials that are compatible with ocular tissue; that is, it does not cause significant or undue detrimental effects when brought into contact with ocular tissue.

The terms “stable,” “stability” or “stabilized” as used herein shall be given their ordinary meaning and shall include products and compositions that enhance the primary, secondary and/or tertiary structure of the polypeptide. In some embodiments, stabilized compositions may have an acceptable percentage of peptide degradation, or aggregation, products after a given period of time. These peptide degradation products can be the result of, for example, oxidation and/or hydrolysis of the peptide.

The terms “peptide”, “polypeptide” and “protein” as used herein, are used interchangeably and shall be given its ordinary meaning. Unless otherwise clear from the context, the noted terms include a polymer having at least two amino acids linked through peptide bonds. The terms thus include oligopeptides, analogs, derivatives, acetylated derivatives, glycosylated derivatives, pegylated derivatives, and the like.

The term “pharmaceutically acceptable salt” shall be given its ordinary meaning and shall include a salt of a compound that does not cause significant irritation to an organism to which it is administered and does not abrogate or substantially reduce the biological activity and properties of the compound. In some embodiments, the salt of the compound may enhance the biological activity and properties of the compound. In other embodiments, the salt may additionally enhance the structural integrity or chemical stability of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid, or phosphoric acid. Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluensulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C₁-C₇ alkylamine, cyclohexylamine, triethanolamine, ethylenediamine, and salts with amino acids such as arginine and lysine. In some embodiments, the polypeptide is an acetate salt.

Provided herein are methods of treating a dry eye-related ocular symptoms by administering a therapeutic amount of a polypeptide to the eye of a subject in need thereof. As used herein, “dry eye-related ocular symptom” has its customary and ordinary meaning as understood by one of ordinary skill in the art in view of the present disclosure. The ocular symptom can include one or more symptoms associated with dry eye disease (or keratoconjunctivitis sicca). Dry eye disease is a multifactorial disease of the ocular surface characterized by a loss of homeostasis of the tear film. It can be accompanied by ocular symptoms, in which tear film instability and hyperosmolarity, ocular surface inflammation and damage, and neurosensory abnormalities play etiological roles. Dry eye-related ocular symptoms can include, without limitation, blurred vision, light sensitivity, sandy or gritty feeling, ocular irritation, ocular pain, burning, or discomfort, ocular stinging, and ocular itching. In some embodiments, the subject does not have Sjögren's Syndrome (Primary or Secondary). As used herein, “moderate/severe” or “moderate to severe” referring to dry eye disease or symptoms are used interchangeably and have their customary and ordinary meanings as understood by one of ordinary skill in the art in view of the present disclosure. In some embodiments, a subject has moderate/severe dry eye and/or dry eye symptoms when the subject meets all of the following criteria in at least one eye.
a) FCS total score ≥4 and <15 in the NEI/Industry Workshop scale,
b) Eye Dryness score of ≥60 using the VAS instantaneous,
c) Anesthetized Schirmer 1 test score ≤5 mm wetting/5 min, and
d) LGCS total score ≥5 using the NEI/Industry Workshop scale (where 0=no staining). As used herein, “severe” referring to dry eye disease or symptoms has its customary and ordinary meanings as understood by one of ordinary skill in the art in view of the present disclosure. In some embodiments, a subject has severe dry eye and/or dry eye symptoms when the subject has Eye Dryness score of ≥70 using the VAS instantaneous measure in at least one eye.

The therapeutic polypeptide of the present disclosure includes lacritin and/or therapeutically active fragments thereof, such as Lacripep™ as described herein. The present Application discloses the surprising finding that the therapeutic effect of Lacripep™ in relieving symptoms of subjects with moderate/severe dry eye disease, and optionally those subject with severe dry eye, can be greater when the polypeptide is administered in a composition at a concentration of not more than 22 μM, (corresponding to about 0.005% w/v), or more preferably at a concentration of 4 μM, (corresponding to about 0.001% w/v), or 1 M, (corresponding to about 0.00025% w/v) than when administered at a higher concentration (e.g., 44 μM corresponding to about 0.01% w/v). In some embodiments, the subject does not have Sjögren's Syndrome (Primary or Secondary).

As described above and herein, several embodiments of the application provide compositions that are stable at room temperature. In several embodiments, the compositions have reduced levels of stabilizers and other additives that may cause undesired side effects, and yet still provide the desired stability. In some embodiments, the composition provides stability in the eye, nasal cavity, mouth, epithelium and other tissues for up to 1, 3, 6, 12, 24 and 48 hours. In some embodiments, the composition is formulated such that some or all of the ingredients do not evaporate, become absorbed, drained or otherwise eliminated after application to the eye or other region, and instead remain stable and active for several hours (e.g., 1-3 hours, 3-6 hours, 6-12 hours, 12-24 hours, and ranges therein). In some embodiments, the composition comprises a peptide, for example Lacripep™ or the other sequences identified herein, where the peptide is applied to the eye, and the peptide is integrated into the lipid layer of the tear covering the eye, or at the interface of the lipid and aqueous components of the tear, where the peptide stabilizes the tear and remains in the tear for a period of at least 1-3 hours, at least 3-6 hours, or at least 12-24 hours, or more than 24 hours. This feature, in several embodiments, is particularly advantageous because it allows an active ingredient (such as a peptide) to remain stable and efficacious for prolonged periods of time. In some embodiments, reduced frequency of administration results in an overall reduced overall burden of ingredients to sensitive areas of the body (such as the eye).

Although peptides are provided in several embodiments herein, other compounds may be used as the active ingredient in addition to a peptide.

Peptides are highly selective and efficacious and, at the same time, relatively safe and well tolerated. Peptides are particularly well-suited for the compositions described herein because peptides may be chemically and physically unstable, relative to certain small-molecule-based therapeutics. For example, peptides are prone to hydrolysis, oxidation, and aggregation. Polypeptide compositions are typically aqueous solutions containing the active peptide along with numerous stabilizers, preservatives, and other agents to maintain the efficacy of the peptide. The stabilizers, preservatives, and other agents may maintain the chemical and/or structural integrity of the polypeptide, thus preserving its efficacy. Certain additives, such as stabilizers and preservatives, may cause undesirable side-effects, including hypersensitivity reactions, itching, and stinging or burning. However, to maximize the shelf-life of the peptide and maintain efficacy, these additives are required in most peptide compositions in amounts that cause undesired results. Even in compositions with all these additives, peptide therapeutics must typically be refrigerated, making transportation difficult, and, even with refrigeration, still have a short shelf-life. Moreover, as the peptides degrade and/or aggregate over time (especially through warming and cooling when taken from cold storage to room temperature for use), the by-products may not only be inactive, they may be toxic and/or immunogenic. Formulators may attempt to increase potency of peptide compositions by increasing the amount of the active peptide in the composition. However, increased peptide concentration also increases the rate of peptide aggregation and inactivation.

Thus, several embodiments herein provide peptide compositions that provide therapeutic amounts of peptides, are stable at room temperature, and contain reduced (e.g., only trace amounts) of stabilizers and/or preservatives, or none at all.

In some embodiments, the peptide is selected from the group consisting of: (a) The amino acid sequence KQFIENGSEFAQKLLKKFS, Ac-KQFIENGSEFAQKLLKKFS-NH₂, or Ac-Lys-Gln-Phe-Ile-Glu-Asn-Gly-Ser-Glu-Phe-Ala-Gln-Lys-Leu-Leu-Lys-Lys-Phe-Ser-NH₂, where “Ac” represents an acetyl group and the C-terminus is amidated (SEQ ID NO: 1), also referred to herein as “Lacripep™”; and, (b) the amino acid sequence KQFIENGSEFAQKLLKKFSLLKPWA, Ac-KQFIENGSEFAQKLLKKFSLLKPWA-NH₂, or Ac-Lys-Gln-Phe-Ile-Glu-Asn-Gly-Ser-Glu-Phe-Ala-Gln-Lys-Leu-Leu-Lys-Lys-Phe-Ser-Leu-Leu-Lys-Pro-Trp-Ala-NH₂, where “Ac” represents an acetyl group and the C-terminus is amidated (SEQ ID NO: 2). In other embodiments, the peptide has the amino acid sequence of one of the following, or a fragment thereof, optionally with the N-terminus acetylated and/or the C-terminus amidated:

```
SEQ ID NO 3
LENGTH: 138
TYPE: PRT
ORGANISM: Homo sapiens
SEQUENCE: 3
Met Lys Phe Thr Thr Leu Leu Phe Leu Ala Ala Val
1 5 10
Ala Gly Ala Leu Val Tyr Ala Glu Asp Ala Ser Ser
                15 20
Asp Ser Thr Gly Ala Asp Pro Ala Gln Glu Ala Gly
25 30 35
Thr Ser Lys Pro Asn Glu Glu Ile Ser Gly Pro Ala
                        40 45
Glu Pro Ala Ser Pro Pro Glu Thr Thr Thr Thr Ala
        50 55 60
Gln Glu Thr Ser Ala Ala Ala Val Gln Gly Thr Ala
                                65 70
Lys Val Thr Ser Ser Arg Gln Glu Leu Asn Pro Leu
                75 80
Lys Ser Ile Val Glu Lys Ser Ile Leu Leu Thr Glu
85 90 95
Gln Ala Leu Ala Lys Ala Gly Lys Gly Met His Gly
                        100 105
Gly Val Pro Gly Gly Lys Gln Phe Ile Glu Asn Gly
        110 115 120
Ser Glu Phe Ala Gln Lys Leu Leu Lys Lys Phe Ser
                                125 130
Leu Leu Lys Pro Trp Ala
                135
SEQ ID NO 4
LENGTH: 119
TYPE: PRT
ORGANISM: Homo sapiens
SEQUENCE: 4
Glu Asp Ala Ser Ser Asp Ser Thr Gly Ala Asp Pro
1 5 10
Ala Gln Glu Ala Gly Thr Ser Lys Pro Asn Glu Glu
                15 20
Ile Ser Gly Pro Ala Glu Pro Ala Ser Pro Pro Glu
25 30 35
Thr Thr Thr Thr Ala Gln Glu Thr Ser Ala Ala Ala
                        40 45
Val Gln Gly Thr Ala Lys Val Thr Ser Ser Arg Gln
        50 55 60
Glu Leu Asn Pro Leu Lys Ser Ile Val Glu Lys Ser
                                65 70
Ile Leu Leu Thr Glu Gln Ala Leu Ala Lys Ala Gly
                75 80
Lys Gly Met His Gly Gly Val Pro Gly Gly Lys Gln
85 90 95
Phe Ile Glu Asn Gly Ser Glu Phe Ala Gln Lys Leu
                        100 105
Leu Lys Lys Phe Ser Leu Leu Lys Pro Trp Ala
        110 115
SEQ ID NO 5
LENGTH: 114
TYPE: PRT
ORGANISM: homo sapiens
SEQUENCE: 5
Asp Ser Thr Gly Ala Asp Pro Ala Gln Glu Ala Gly
1 5 10
Thr Ser Lys Pro Asn Glu Glu Ile Ser Gly Pro Ala
                15 20
Glu Pro Ala Ser Pro Pro Glu Thr Thr Thr Thr Ala
25 30 35
Gln Glu Thr Ser Ala Ala Ala Val Gln Gly Thr Ala
                        40 45
Lys Val Thr Ser Ser Arg Gln Glu Leu Asn Pro Leu
        50 55 60
Lys Ser Ile Val Glu Lys Ser Ile Leu Leu Thr Glu
                                65 70
Gln Ala Leu Ala Lys Ala Gly Lys Gly Met His Gly
                75 80
Gly Val Pro Gly Gly Lys Gln Phe Ile Glu Asn Gly
85 90 95
Ser Glu Phe Ala Gln Lys Leu Leu Lys Lys Phe Ser
                        100 105
Leu Leu Lys Pro Trp Ala
        110
SEQ ID NO 6
LENGTH: 114
TYPE: PRT
ORGANISM: Homo sapiens
SEQUENCE: 6
Glu Asp Ala Ser Ser Asp Ser Thr Gly Ala Asp Pro
1 5 10
Ala Gln Glu Ala Gly Thr Ser Lys Pro Asn Glu Glu
                15 20
Ile Ser Gly Pro Ala Glu Pro Ala Ser Pro Pro Glu
25 30 35
Thr Thr Thr Thr Ala Gln Glu Thr Ser Ala Ala Ala
                        40 45
Val Gln Gly Thr Ala Lys Val Thr Ser Ser Arg Gln
        50 55 60
Glu Leu Asn Pro Leu Lys Ser Ile Val Glu Lys Ser
                                65 70
Ile Leu Leu Thr Glu Gln Ala Leu Ala Lys Ala Gly
                75 80
Lys Gly Met His Gly Gly Val Pro Gly Gly Lys Gln
85 90 95
Phe Ile Glu Asn Gly Ser Glu Phe Ala Gln Lys Leu
                        100 105
Leu Lys Lys Phe Ser Leu
        110
SEQ ID NO 7
LENGTH: 109
TYPE: PRT
ORGANISM: homo sapiens
SEQUENCE: 7
Glu Asp Ala Ser Ser Asp Ser Thr Gly Ala Asp Pro
1 5 10
Ala Gln Glu Ala Gly Thr Ser Lys Pro Asn Glu Glu
                15 20
Ile Ser Gly Pro Ala Glu Pro Ala Ser Pro Pro Glu
25 30 35
Thr Thr Thr Thr Ala Gln Glu Thr Ser Ala Ala Ala
                        40 45
Val Gln Gly Thr Ala Lys Val Thr Ser Ser Arg Gln
        50 55 60
Glu Leu Asn Pro Leu Lys Ser Ile Val Glu Lys Ser
                                65 70
Ile Leu Leu Thr Glu Gln Ala Leu Ala Lys Ala Gly
                75 80
Lys Gly Met His Gly Gly Val Pro Gly Gly Lys Gln
85 90 95
Phe Ile Glu Asn Gly Ser Glu Phe Ala Gln Lys Leu
                        100 105
Leu
SEQ ID NO 8
LENGTH: 104
TYPE: PRT
ORGANISM: homo sapiens
SEQUENCE: 8
Glu Asp Ala Ser Ser Asp Ser Thr Gly Ala Asp Pro
1 5 10
Ala Gln Glu Ala Gly Thr Ser Lys Pro Asn Glu Glu
                15 20
Ile Ser Gly Pro Ala Glu Pro Ala Ser Pro Pro Glu
25 30 35
Thr Thr Thr Thr Ala Gln Glu Thr Ser Ala Ala Ala
                        40 45
Val Gln Gly Thr Ala Lys Val Thr Ser Ser Arg Gln
        50 55 60
Glu Leu Asn Pro Leu Lys Ser Ile Val Glu Lys Ser
                                65 70
Ile Leu Leu Thr Glu Gln Ala Leu Ala Lys Ala Gly
                75 80
Lys Gly Met His Gly Gly Val Pro Gly Gly Lys Gln
85 90 95
Phe Ile Glu Asn Gly Ser Glu Phe
                        100
SEQ ID NO 9
LENGTH: 14
TYPE: PRT
ORGANISM: homo sapiens
SEQUENCE: 9
Asn Gly Ser Glu Phe Ala Gln Lys Leu Leu Lys Lys
1 5 10
Phe Ser
```

In several embodiments, the peptide is represented by the amino acid sequence Ac-KQFIENGSEFAQKLLKKFS-NH₂ or Ac-Lys-Gln-Phe-Ile-Glu-Asn-Gly-Ser-Glu-Phe-Ala-Gln-Lys-Leu-Leu-Lys-Lys-Phe-Ser-NH₂, where “Ac” represents an acetyl group and the C-terminus is amidated (SEQ ID NO: 1). In some embodiments, the peptide is Lacripep™. In some embodiments, the peptide is any one or more of SEQ. IDs 1-9.

### Buffers and pH

Buffers stabilize the pH of a solution, i.e., resist changes in pH when acidic or alkaline materials are added to the solution. Suitable buffers for use in the present composition include, but are not limited to, glycine hydrochloride, sodium acetate, phosphate buffered saline (PBS) (including mono- and dihydrogen phosphate salts), citrate buffer (citric acid and sodium citrate), phosphate-citrate buffer, tris(hydroxymethyl)aminomethane (Tris), carbonate buffers (sodium carbonate and sodium bicarbonate), borate buffers, and combinations thereof.

In some embodiments, the buffer comprises one or more of sodium acetate, phosphate buffered saline (PBS), citrate buffer (citric acid and sodium citrate), and phosphate-citrate buffer. In some embodiments, the buffer is selected from the group consisting of sodium acetate, phosphate buffered saline (PBS), citrate buffer (citric acid and sodium citrate), and phosphate-citrate buffer.

In some embodiments, the amount of buffer is limited to less than 0.1, 0.2, 0.3, or, 0.4%, or within a range defined by any two of the preceding values.

In an embodiment, the buffer is a citrate buffer (citric acid and sodium citrate). In an embodiment, the only buffer is a citrate buffer, and no other buffering agent is present in the composition.

In some embodiments the pH of the composition is 6.0 to 7.4; 6.1 to 7.3; 6.2 to 7.2; 6.3 to 7.1; 6.4 to 7.0; 6.5 to 6.9; 6.6 to 6.8; or any pH in between.

In some embodiments the pH of the composition is, or is about, 6.0; 6.1; 6.2; 6.3; 6.4; 6.5; 6.6; 6.7; 6.8; 6.9; 7; 7.1; 7.2; 7.3; 7.4, or a range defined by any two of the preceding values, for example, from 6.0-7.0, 6.2-6.5, 6.3-6.5, 6.0-6.5, 6.3-6.7, or 6.3-7.0. In some embodiments, the pH of the composition is, or is about, 6.2, 6.3, 6.4, 6.5, 6.6, 6.7, or 6.8. In some embodiments, the pH of the composition is, or is about, 6.0, 6.1, 6.2, 6.3, 6.4, 6.5. In some embodiments, the pH of the composition is, or is about, 6.2, 6.3, 6.4, 6.5, 6.6, 6.7, or 6.8.

The pH of the composition can be adjusted as necessary by the addition of solutions of an acid or a base. Any acid or base whose conjugate is ophthalmically acceptable may be used. Acids include for example hydrochloric acid, bases include for example sodium and potassium hydroxides.

In some embodiments, the pH is measured using the USP<791> methodology.

### Chelating Agents

In some embodiments, the composition further comprises one or more chelating agents. In some embodiments, the chelating agents are selected from the group consisting of ethylenediaminetetraacetic acid, edetate disodium (EDTA), ethylenediamine, amino acids such as glutamic acid and histidine, organic diacids such as oxalic acid, malonic acid, succinic acid, and the like, 3-dimercaptopropanesulfonic acid (DMPS), alpha lipoic acid (ALA), 2,3-dimercaptopropanesulfonic acid (DMPS), thiamine tetrahydrofurfuryl disulfide (TTFD), penicillamine, dimercaptosuccinic acid (DMSA), combinations thereof, and pharmaceutically acceptable salts of the foregoing.

In some embodiments, the chelating agent, as a non-limiting example EDTA, or a pharmaceutically acceptable salt thereof, is present at 0.0001% to 0.1%; 0.0005% to 0.05%; 0.0006% to 0.04%; 0.0007% to 0.003%; 0.0008% to 0.002%; 0.0009% to 0.001%; or any value contained therein or ranges therein. In some embodiments, the chelating agent is present at an amount that is, or is less than, 0.1%; 0.09%; 0.08%; 0.07%; 0.06%; 0.05%; 0.04%; 0.03%; 0.02%; 0.01%; 0.0090%; 0.008%; 0.007%; 0.006%; 0.005%; 0.004%; 0.003%; 0.002%; 0.001%; 0.0009%; 0.0008%; 0.0007%; 0.0006%; 0.0005%; 0.0004%; 0.0003%; 0.0002%; or 0.0001%, or is within a range defined by any two of the preceding values.

In some embodiments, the chelating agent, such as EDTA or others, or a pharmaceutically acceptable salt thereof, is present at less than about 0.05% or less than about 0.005% (e.g., at about 0.001%).

### Stabilizing Agents

Buffers and chelators can stabilize peptide ingredients of compositions by maintaining pH and reducing metal ion mediated degradation of the peptides. In some embodiments, the composition further comprises one or more peptide stabilizing agents in addition to a buffer and/or a chelating agent. In some embodiments, the one or more stabilizing agents in addition to a buffer and/or chelating agent are selected from the group consisting of disaccharides, polysaccharides (e.g., hyaluronic acid), polyols, sugar alcohols, amino acids, proteins (e.g., serum albumin), and combinations thereof. In some embodiments, non-limiting examples of stabilizers include trehalose, sucrose, mannitol, sorbitol, polysorbate 20, polysorbate 80, histidine, glycine, and arginine, and combinations thereof. In an embodiment the composition does not include a stabilizer in addition to a buffering agent and/or a chelator.

### Polypeptide Degradation

Polypeptides are prone to physical and chemical degradation, for example, aggregation, shearing, oxidation, deamidation, and hydrolysis. Indeed, liquid peptide compositions have a high risk for physical and chemical instability during manufacturing and storage. Reducing polypeptide degradation is particularly important for dilute peptide formulations, which initially contain very small amounts of a particular peptide. Loss of even miniscule amounts of the initial small amount can significantly impact the efficacy of the composition.

In some embodiments, composition stability is determined by high-performance liquid chromatography (HPLC). In some embodiments, composition stability is determined by high-performance liquid chromatography-mass spectrometry (HPLC-MS).

In some embodiments, composition stability is determined after a sealed container of the composition has been in the dark, or exposed to light, at room temperature for days, weeks or months (e.g., 1-24 days or months, e.g., 1, 2, 3, 4, 5, 6, 7, 8, 9, 10, 11, 12, 13, 14, 15, 16, 17, 18, 19, 20, 21, 22, 23, 24 days or months).

In some embodiments, composition stability is determined after a sealed container of the composition has been in the dark, or exposed to light, at 2 to 8° C., for example 5° C., or any value in between, for days, weeks or months, (e.g., 1-24 days or months, e.g., 1, 2, 3, 4, 5, 6, 7, 8, 9, 10, 11, 12, 13, 14, 15, 16, 17, 18, 19, 20, 21, 22, 23, 24 days or months)

In some embodiments, composition stability is determined after a sealed container of the composition has been in the dark, or exposed to light, at −10 to −30° C., for example −25° C., or any value in between, for days, weeks or months, (e.g., 1-24 days or months, e.g., 1, 2, 3, 4, 5, 6, 7, 8, 9, 10, 11, 12, 13, 14, 15, 16, 17, 18, 19, 20, 21, 22, 23, 24 days or months)

In some embodiments, composition stability is determined after a sealed container of the composition has been in the dark, or exposed to light, and moved from 2 to 8° C. (storage), or any value in between, to room temperature for 5 minutes, either one, two, or three times per day, for 1-60 days.

In some embodiments, the composition provides at least 99.99%, 99.95%, 99.9%, 99%; 98%; 97%; 96%; 95%; 94%; 93%; 92%; 91%; 90%; 89%; 88%; 87%; 86%; 85%; 84%; 83%; 82%; 81%; 80%; 79%; 78%; 77%; 76%; 75%; 74%; 73%; 72%; 71%; 70%; or any value in between, of the original amount or activity of the polypeptide, or a pharmaceutically acceptable salt thereof, in an intact, non-degraded or non-aggregated form, following exposure to one or more of the conditions described above and herein. In a some embodiments, the amount or activity of the intact polypeptide, or a pharmaceutically acceptable salt thereof, is at least 80%, 85%, 90% or 95% of the original amount. In some embodiments, the amount or activity of intact polypeptide, or a pharmaceutically acceptable salt thereof, is at least 97% of the original amount.

In some embodiments, the composition comprises not more than 30%; 29%; 28%; 27%; 26%; 25%; 24%; 23%; 22%; 21%; 20%; 19%; 18%; 17%; 16%; 15%; 14%; 13%; 12%; 11%; 10%; 9%; 8%; 7%; 6%; 5%; 4%; 3%; 2%; 1%; of a peptide aggregation product or peptide degradation product, or is within a range defined by any two of the preceding values, following exposure to one or more of the conditions described above and herein. In some embodiments, the composition comprises not more than about 15%, or not more than 20%, inactive peptide.

In some embodiments, the composition comprises not more than 30%; 29%; 28%; 27%; 26%; 25%; 24%; 23%; 22%; 21%; 20%; 19%; 18%; 17%; 16%; 15%; 14%; 13%; 12%; 11%; 10%; 9%; 8%; 7%; 6%; 5%; 4%; 3%; 2%; 1%; of the total amounts of peptide degradation products and peptide aggregation products, or is within a range defined by any two of the preceding values, following exposure to one or more of the conditions described above and herein.

In some embodiments, the composition comprises very low levels of buffer, in combination with very low levels of a chelator. In some embodiments, the buffer is a citrate buffer and the chelator is EDTA. The combination of low levels of citrate buffer (e.g., 0.012% to 0.020%) and EDTA (e.g., 0.0005% to 0.005%), provide the surprising and unexpected benefit of stabilizing compositions containing low levels of a peptide (e.g., 0.001 to 0.01%). Such stabilized compositions provide advantages in manufacturing, transportation, storage, and use of the peptide compositions by decreasing peptide aggregation and degradation, thus maintaining the efficacy of peptide compositions and reducing buildup of undesired breakdown products in the composition.

In some embodiments, the stabilized composition reduces the rate of formation of breakdown and/or aggregation products.

In some embodiments, the peptide is Lacripep™. In some embodiments, the stabilized composition comprises less than about 5%, 4%, 3%, 2%, or about 1% total degradation products. In some embodiments, the stabilized composition comprises not more than 0.25%, 0.5%, 0.75%, 1.0%, 1.25%, 1.5%, 1.75%, or 2.0% of any single degradation product. In some embodiments, the stabilized composition comprises less than about 5%, 4%, 3%, 2%, or about 1% total degradation products and not more than 0.25%, 0.5%, 0.75%, 1.0% c, 1.25%, 1.5%, 1.75%, or 2.0% of any single degradation product.

In some embodiments, the aggregation products include dimers, trimers, tetramers, or larger-order peptide aggregates.

### Preservatives

In some embodiments, the composition further comprises one or more preservatives to prevent the growth of microbes in the composition. In some embodiments, the composition further comprises one or more preservatives to maintain the sterility of the composition. In some embodiments, the composition further comprises one or more preservatives to prevent the growth of microbes and maintain the sterility of the composition. However, in many embodiments, the preservative is provided in reduced amounts. In some embodiments, the one or more preservatives are selected from the group consisting of benzalkonium chloride, cetylpyridinium chloride, chlorobutanol, benzododecinium bromide, methylparaben, propylparaben, phenylethyl alcohol, sodium perborate, edentate disodium, chlorobutanol, sorbic acid, benzethonium chloride, sodium acetate, polyquaternium-1, phenylmercuric nitrate, phenylmercury borate, sodium propionate, chlorhexidine, thimerosal, and combinations thereof. In some embodiments, the composition does not contain a preservative. In some embodiments, the composition does not contain detectable levels of a preservative. In some embodiments, the polypeptide can be self-preserving, i.e., no additional preservatives are necessary to maintain sterility of the composition.

In some embodiments, the preservative is present at 0.0001% to 1%; 0.01% to 0.9%; 0.05% to 0.8%; 0.1% to 0.7%; 0.2% to 0.3%; 0.4% to 0.5%, or any value contained therein. In some embodiments, the preservative is present in an amount that is, or is less than, 1%; 0.9%; 0.8%; 0.7%; 0.6%; 0.5%; 0.4%; 0.3%; 0.2%; 0.1%; 0.09%; 0.08%; 0.07%; 0.06%; 0.05%; 0.04%/c; 0.03%; 0.02%; 0.01%; 0.009%; 0.008%; 0.007%; 0.006%; 0.005%; 0.004%; 0.003%; 0.002%; or 0.001%, or is within a range defined by any two of the preceding values.

In some embodiments, the composition is sterile. In some embodiments, the composition is manufactured from sterile ingredients in an aseptic environment. In some embodiments, the composition is sterilized just prior to packaging. In some embodiments, the composition is sterilized by one or more of the following (1) addition of one or more quaternary ammonium chlorides to the composition; (2) exposing the composition to ionizing radiation; (3) filtering the composition; (4) exposing the composition to ionizing radiation after packaging; and any combination of the foregoing. In some embodiments, filtering comprises passing the composition through a filter (including but not limited to a 0.22 micron filter with a polyvinyldifluoride or other suitable membrane (e.g., polyethersulfone).

In some embodiments, the peptide is provided in a bacteriostatic and/or bactericidal amount. In some embodiments, the amount of peptide provided in the composition is bacteriostatic and/or bactericidal when one, two or three drops of the composition are administered to the surface of the eye. In some embodiments, the peptide is bacteriostatic and/or bactericidal for Gram-positive and/or Gram-negative bacteria, for example, when administered to the eye. In some embodiments the amount of peptide in the composition is sufficient to inhibit bacterial growth by at least 20%, 30%, 40%, 50%, 60%, 70%, 80% or 90% relative to a control composition not containing the peptide in a standard bacteriological assay. In some embodiments, the bacteria in the bacteriological assay are selected from P. aeruginoa, E coli, S epidermis, S. aureus, or combinations thereof. In some embodiments, the bacteriological assay is selected from a bacterial growth assay, SYTOX Green assay, a well diffusion assay, a broth or agar dilution assay, a time-kill test, antimicrobial gradient assay, a ATP-bioluminescence assay, or a propidium-iodide flow cytometry assay. In some embodiments, the peptide provided in a bacteriostatic and/or bactericidal amount is Lacripep™.

In some embodiments, the bacteriological assay is a USP Section <51> assay or FDA-mandated assay. For example, the original product containers, containing the peptide solution, and inoculate each container with one of the prepared and standardized inoculums (e.g., P. aeruginoa, E. coli, S. epidermis, S. aureus, or combinations thereof) and mix. The volume of the suspension inoculums should be about 0.5% to 1.0% of the volume of the product, and the concentration of the test preparation immediately after inoculation is between 1×10⁵ and 1×10⁶ colony forming organisms (CFU) per mL of product (as measured by, for example, the plate count method, or another microbial enumeration test).

The inoculated containers are incubated at between 22.5±2.5° C. in a controlled environment and sampled at specified intervals, for example, 7, 14, and 28 days. Any change in appearance is recorded, and the CFU/mL are determined, at each sampling. The change in log₁₀ values of CFU/mL provides the change over time in terms of log reductions. The product provides not less than 1.0 log reduction from the initial calculated count at 7 days, not less than 3.0 log reduction from the initial count at 14 days, and no increase from the 14 day count at 28 days for bacteria, and no increase from the initial count of yeast and molds. In some embodiments, the peptide provided in a bacteriostatic and/or bactericidal amount is Lacripep™.

### Surfactants

In some embodiments, the composition further comprises one or more surfactants. In some embodiments, the one or more surfactants are selected from detergents, wetting agents, emulsifiers, foaming agents, dispersants, and combinations thereof.

In some embodiments, the surfactant is an anionic surfactant. Anionic surfactants contain anionic functional groups at their head, such as sulfate, sulfonate, phosphate, and carboxylates. In some embodiments, the surfactant is a sulfate, sulfonate, or phosphate ester, e.g., a sulfate ester. In some embodiments, the surfactant is selected from the group comprising or consisting of ammonium lauryl sulfate and sodium lauryl sulfate, e.g., sodium lauryl sulfate (also called SDS, sodium dodecyl sulfate). In some embodiments, the surfactant is an alkyl-ether sulfate, such as selected from the group comprising or consisting of sodium laureth sulfate (also known as sodium lauryl ether sulfate), and sodium myreth sulfate. In some embodiments, the surfactant is a docusate, such as dioctyl sodium sulfosuccinate, perfluorooctanesulfonate (PFOS), perfluorobutanesulfonate, linear alkylbenzene sulfonates (LABs). In some embodiments, the surfactant is a carboxylate, such as alkyl carboxylates (soaps), for instance sodium stearate; sodium lauroyl sarcosinate and carboxylate-based fluorosurfactants such as perfluorononanoate, perfluorooctanoate (PFOA or PFO). In some embodiments, the polypeptide contributes to the surfactant properties of the composition.

In some embodiments, the surfactant is a cationic surfactant, of which the charge can be pH dependent, such as primary, secondary or tertiary amines, for instance octenidine dihydrochloride; or may comprise permanently charged quaternary ammonium cations, such as alkyltrimethylammonium salts, for instance cetyl trimethylammonium bromide (CTAB) or cetyl trimethylammonium chloride (CTAC); cetylpyridinium chloride (CPC); benzalkonium chloride (BAC); benzethonium chloride (BZT); 5-Bromo-5-nitro-1,3-dioxane; dimethyldioctadecylammonium chloride; or dioctadecyldimethylammonium bromide (DODAB). In some embodiments, the surfactant is a zwitterionic surfactant (i.e. having both cationic and anionic centers attached to the same molecule). The cationic part may be based on primary, secondary, or tertiary amines or quaternary ammonium cations. The anionic part can be more variable and include sulfonates, as in CHAPS (3-[(3-Cholamidopropyl)dimethylammonio]-1-propanesulfonate). Other anionic groups are sultaines illustrated by cocamidopropyl hydroxysultaine; betaines, e.g., cocamidopropyl betaine; phosphates, e.g. lecithin. In some embodiments, the surfactant may be a non-ionic surfactant (not charged).

Many long chain alcohols exhibit some surfactant properties, and are provided herein as part of a composition in some embodiments. Prominent among these are the fatty alcohols cetyl alcohol, stearyl alcohol, and cetostearyl alcohol (consisting predominantly of cetyl and stearyl alcohols), and oleyl alcohol. Other surfactants include cocamide MEA, cocamide DEA, dodecyldimethylamine oxide, and polyethoxylated tallow amine (POEA). Examples of non-ionic surfactants include polyoxyethylene glycol alkyl ethers, such as octaethylene glycol monododecyl ether or pentaethylene glycol monododecyl ether; polyoxypropylene glycol alkyl ethers; glucoside alkyl ethers, such as decyl glucoside, lauryl glucoside, or octyl glucoside; polyoxyethylene glycol octylphenol ethers, such as Triton X-100; polyoxyethylene glycol alkylphenol ethers, such as Nonoxynol-9; glycerol alkyl esters, such as glyceryl laurate; polyoxyethylene glycol sorbitan alkyl esters (polysorbate); sorbitan alkyl esters (Spans); block copolymers of polyethylene glycol and polypropylene glycol, or Poloxamers.

In some embodiments, the composition may contain one or more ingredients found in artificial tears in amounts known in the art, including but not limited to: carboxymethyl cellulose, polyvinyl alcohol, hydroxypropyl methylcellulose (a.k.a. HPMC or hypromellose), hydroxypropyl cellulose, hydroxyethyl cellulose (HEC), and hyaluronic acid (a.k.a. hyaluronan, HA), and combinations thereof. In some embodiments, the composition does not contain any of the preceding artificial tear ingredients.

In some embodiments, the surfactant is another peptide or protein. In some embodiments, as a non-limiting example, the surfactant is human serum albumin. In some embodiments, as another non-limiting example, the surfactant is Lacripep™.

In several embodiments, the surfactant is tyloxapol (formaldehyde; oxirane; 4-(2,4,4-trimethylpentan-2-yl)phenol). In an embodiment, the only surfactant is tyloxapol, and no other surfactant agent is present in the composition.

In some embodiments, the surfactant, as a non-limiting example tyloxapol, is present at 0.01% to 1%; 0.05% to 0.9%; 0.1% to 0.8%; 0.2% to 0.7%; 0.3% to 0.6%; 0.4% to 0.5%, or any value contained therein. In some embodiments, the surfactant is present in an amount that is, or is less than, 1%; 0.9%; 0.8%; 0.7%; 0.6%; 0.5%; 0.4%; 0.3%; 0.22%; 0.1%; 0.09%; 0.08%; 0.07%; 0.06%; 0.05%; 0.04%; 0.03%; 0.02%; 0.01%; 0.009%; 0.008%; 0.007%; 0.006%; 0.005%; 0.004%; 0.003%; 0.002%; or 0.001%, or is within a range defined by any two of the preceding values.

In some embodiments, the composition includes tyloxapol at, or at about, 0.0001%, 0.0002%, 0.0005%, 0.001%, 0.002%, 0.005%, 0.01%, 0.02%, or 0.05% (w/v), or at a percentage in a range defined by any two of the preceding values (e.g., 0.0001-0.05%, 0.0001-0.02%, 0.001-0.01%, 0.0001-0.005%, 0.0002-0.002%, 0.0005-0.002%, 0.0002-0.001%, 0.001-0.01%, 0.001-0.05% or 0.001-0.005%, etc.). In some embodiments, tyloxapol is present in the liquid composition at 0.0001-0.002% (w/v). In some embodiments, tyloxapol is present in the liquid composition at 0.0005-0.005% (w/v). In some embodiments, tyloxapol is present in the liquid composition at 0.001-0.01% (w/v). In some embodiments, the tyloxapol is present in the liquid composition at about 0.001% (w/v). In some embodiments, the tyloxapol is present in the liquid composition at about 0.01% (w/v). In some embodiments, the tyloxapol is present in the liquid composition at about 0.05% (w/v).

In some embodiments, the composition does not contain a surfactant. In some embodiments, the composition does not contain detectable levels of a surfactant.

### Tonicity Agents and Osmolality

In some embodiments, the composition further comprises one or more tonicity agents. Such tonicity agents are in addition to any polypeptide or buffer that has tonicity-modifying effects. In some embodiments, the one or more tonicity agents are selected from propylene glycol, polyethylene glycols, sodium chloride, potassium chloride, magnesium chloride, calcium chloride, simple sugars such as dextrose, fructose, galactose, and/or simple polyols such as the sugar alcohols mannitol, sorbitol, xylitol, lactitol, isomaltitol, maltitol, hydrogenated starch hydrolysates, glycerin, and combinations thereof.

In some embodiments, the one or more tonicity agents are selected from sodium chloride, potassium chloride, magnesium chloride, calcium chloride, dextrose, mannitol, and combinations thereof.

In some embodiments, the tonicity agent is sodium chloride. In some embodiments, the sodium chloride is present at 0.019% to 1%; 0.05% to 0.9%; 0.1% to 0.8%; 0.2% to 0.75%; 0.3% to 0.7%; 0.4% to 0.6%; or any value contained therein. In some embodiments, the sodium chloride is present at an amount that is, or is about, 1%; 0.95%; 0.9%; 0.85%; 0.8%; 0.75%; 0.7%; 0.65%; 0.6%; 0.55%; 0.5%; 0.45%; 0.4%; 0.35%; 0.3%; 0.25%; 0.2%; 0.15%; 0.1%; 0.09%; 0.08%; 0.07%; 0.06%; 0.05%; 0.04%; 0.03%; 0.02%; or 0.01%; or is within a range defined by any two of the preceding values.

In some embodiments, the only tonicity agent is sodium chloride, and no other tonicity agent is present in the composition.

In some embodiments, a tonicity agent, as a non-limiting example sodium chloride, is added to the composition to adjust the osmolality to a desired level. In some embodiments, the osmolality of the composition is about 150 to about 400 mOsm/kg; about 170 to about 380 mOsm/kg; about 190 to about 360 mOsm/kg; about 210 to about 340 mOsm/kg; about 230 to about 320 mOsm/kg; about 250 to about 300 mOsm/kg; about 270 to about 280 mOsm/kg; or any value in between. In some embodiments, the osmolality of the composition is about 250 to about 350 mOsm/kg; about 260 to about 340 mOsm/kg; about 270 to about 330 mOsm/kg; about 280 to about 320 mOsm/kg; about 290 to about 310 mOsm/kg; or any value in between. In some embodiments, the osmolality of the composition is about 150 to about 300 mOsm/kg; about 150 to about 250 mOsm/kg; about 160 to about 230 mOsm/kg; about 170 to about 220 mOsm/kg; about 180 to about 220 mOsm/kg; about 190 to about 210 mOsm/kg; about 190 to about 200 mOsm/kg; about 170 to about 210 mOsm/kg; about 180 to about 200 mOsm/kg; or any value in between.

In some embodiments, the osmolality of the composition is, or is about, 150 mOsm/kg; 160 mOsm/kg; 170 mOsm/kg; 180 mOsm/kg; 190 mOsm/kg; 200 mOsm/kg; 210 mOsm/kg; 220 mOsm/kg; 230 mOsm/kg; 240 mOsm/kg; 250 mOsm/kg; 260 mOsm/kg; 270 mOsm/kg; 280 mOsm/kg; 290 mOsm/kg; 300 mOsm/kg; 310 mOsm/kg; 320 mOsm/kg; 330 mOsm/kg; 340 mOsm/kg; or 350 mOsm/kg, or is within a range defined by any two of the preceding values

In some embodiments, the osmolality of the composition is about 280 mOsm/kg to about 320 mOsm/kg. In some embodiments, the osmolality of the composition is about 300 mOsm/kg. In some embodiments, NaCl is used to adjust the osmolality of the solution to the desired level. In an embodiment, the composition is, or is about, isotonic with human tears.

In some embodiments, the osmolality is measured using the USP<785> methodology.

### Polypeptides and Other Ingredients

In some embodiments, the polypeptide, or a pharmaceutically acceptable salt thereof, has 10 to 150 amino acids; 10 to 50 amino acids; 100 to 150 amino acids; 30 to 70 amino acids; or any number contained therein. In some embodiments, the polypeptide, or a pharmaceutically acceptable salt thereof, has 10 to 30 amino acids; 11 to 29 amino acids; 12 to 28 amino acids; 13 to 27 amino acids; 14 to 26 amino acids; 15 to 25 amino acids; 16 to 24 amino acids; 17 to 23 amino acids; 18 to 22 amino acids; 19 to 21 amino acids; or any number contained therein. In some embodiments, the polypeptide, or a pharmaceutically acceptable salt thereof, is, or is about, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length, or a range defined by any two of the preceding values.

In some embodiments, the C-terminus of the polypeptide, or a pharmaceutically acceptable salt thereof, is amidated. In some embodiments, the N-terminus of the polypeptide, or a pharmaceutically acceptable salt thereof, is acetylated. In some embodiments, one or more side chains of the polypeptide, or a pharmaceutically acceptable salt thereof, are acetylated. In some embodiments, one or more side chains of the polypeptide, or a pharmaceutically acceptable salt thereof, are amidated. In some embodiments, the N-terminus of the polypeptide, or a pharmaceutically acceptable salt thereof, is acetylated and the C-terminus of the polypeptide, or a pharmaceutically acceptable salt thereof, is amidated.

In some embodiments, the polypeptide, or a pharmaceutically acceptable salt thereof, comprises, consists or consists essentially of the amino acid sequence: Ac-Lys-Gln-Phe-Ile-Glu-Asn-Gly-Ser-Glu-Phe-Ala-Gln-Lys-Leu-Leu-Lys-Lys-Phe-Ser-Leu-Leu-Lys-Pro-Trp-Ala-NH₂ (SEQ ID NO: 2), where “Ac” represents an acetyl group and the C-terminus is amidated (indicated by “NH₂”). In some embodiments, the polypeptide, or a pharmaceutically acceptable salt thereof, comprises the amino acid sequence: Ac-Lys-Gln-Phe-Ile-Glu-Asn-Gly-Ser-Glu-Phe-Ala-Gln-Lys-Leu-Leu-Lys-Lys-Phe-Ser-NH₂ (SEQ ID NO: 1), where “Ac” represents an acetyl group and the C-terminus is amidated (indicated by “NH₂”). In some embodiments, the polypeptide, or a pharmaceutically acceptable salt thereof, comprises, consists, or consists essentially of a sequence selected from the group of SEQ ID NOs: 3-9, or fragments, or pharmaceutically acceptable salts thereof.

In some embodiments, the amount of polypeptide, or a pharmaceutically acceptable salt thereof, in the composition is, or is about, 0.0001% to 0.005%; 0.0005% to 0.005%; or 0.001% to 0.005%. In an embodiment, the polypeptide, or a pharmaceutically acceptable salt thereof, is present in the composition at about 0.003% to 0.09% (e.g., 0.005%, 0.010%, 0.02%, 0.03% and ranges thereof).

In some embodiments, the polypeptide, or a pharmaceutically acceptable salt thereof, is present in the composition in an amount that is, is about, is more than, or is less than, 0.0001, 0.00025, 0.0005, 0.00075, 0.001, 0.002, 0.003, 0.004, 0.005, or 0.006%, or a range defined by any two of the preceding values. In some embodiments, the composition includes the polypeptide or a pharmaceutically acceptable salt thereof at, or at about, 0.0001%, 0.0002%, 0.00025%, 0.0003%, 0.0005%, 0.00075%, 0.001%, 0.002%, 0.005%, or 0.006% (w/v), or at a percentage in a range defined by any two of the preceding values (e.g., 0.00025-0.006%, 0.00025-0.005%, 0.00025-0.001%, 0.0001-0.001%, 0.0001-0.002%, or 0.001-0.005%). In some embodiments, the composition includes the polypeptide or a pharmaceutically acceptable salt thereof at 0.00025-0.005% (w/v). In some embodiments, the composition includes the polypeptide or a pharmaceutically acceptable salt thereof at 0.0001-0.002% (w/v). In some embodiments, the composition includes the polypeptide or a pharmaceutically acceptable salt thereof at about 0.00025% (w/v). In some embodiments, the composition includes the polypeptide or a pharmaceutically acceptable salt thereof at about 0.001% (w/v). In some embodiments, the composition includes the polypeptide or a pharmaceutically acceptable salt thereof at about 0.005% (w/v). In some embodiments, the amount of polypeptide of pharmaceutically acceptable salt thereof is, is about, is at least, is at least about, is not more than, is not more than about, 0.1, 0.5, 1.0, 1.5, 2.0, 2.5, 3.0, 3.5, 4.0, 4.5, 5.0, 10, 15, 20, 21, 22, 23, 24, or 25 M, or a range defined by any two of the preceding values (e.g., 0.1-25, 0.1-6.0, 0.1-4.0, 1.0-5.0, 1.0-22, or 1.0-4.0 μM). In some embodiments, the composition includes the polypeptide or a pharmaceutically acceptable salt thereof at about 1.0 μM. In some embodiments, the composition includes the polypeptide or a pharmaceutically acceptable salt thereof at about 4.0 μM. In some embodiments, the composition includes the polypeptide or a pharmaceutically acceptable salt thereof at about 22 μM. In some embodiments the polypeptide or a pharmaceutically acceptable salt thereof has a sequence consisting, or consisting essentially of, Ac-Lys-Gln-Phe-Ile-Glu-Asn-Gly-Ser-Glu-Phe-Ala-Gln-Lys-Leu-Leu-Lys-Lys-Phe-Ser-NH₂ (SEQ ID NO: 1), wherein “Ac” represents an acetyl group and the C-terminus is amidated.

In some embodiments, the composition includes: 0.0001-0.005% (w/v) of a polypeptide or a pharmaceutically acceptable salt thereof, the polypeptide having a sequence consisting, or consisting essentially of Ac-Lys-Gln-Phe-Ile-Glu-Asn-Gly-Ser-Glu-Phe-Ala-Gln-Lys-Leu-Leu-Lys-Lys-Phe-Ser-NH₂ (SEQ ID NO: 1), wherein “Ac” represents an acetyl group and the C-terminus is amidated; 0.01-0.6% (w/v) of a buffer; 0.0005-0.01% (w/v) disodium EDTA; 0.0001-0.02% or 0.0001-0.06% (w/v) tyloxapol; and about 0.1-1.0% NaCl; wherein the pH of the composition is about 6.0 to about 7.0, and the osmolality is 170-210 mOsm/kg. In some embodiments, the composition is a sterile aqueous composition.

In some embodiments, the composition is a sterile aqueous composition comprising, consisting or consisting essentially of about 0.005%±0.0005% of a polypeptide, such as Lacripep™ (SEQ ID NO. 1) or the other peptides identified herein, or a pharmaceutically acceptable salt thereof; about 0.0098%±0.001% anhydrous citric acid; about 0.279%±0.028% sodium citrate dihydrate; about 0.001%±0.0001% disodium EDTA; about 0.001%±0.0001% tyloxapol; about 0.50%±0.05% NaCl; wherein the pH of the composition is adjusted using NaOH or HCl to be about 6.2 to about 6.8, and the osmolality is 170-210 mOsm/kg. In some embodiments, the composition is a sterile aqueous composition comprising, consisting or consisting essentially of about 0.005%±0.0005% of a polypeptide, such as Lacripep™ (SEQ ID NO. 1) or the other peptides identified herein, or a pharmaceutically acceptable salt thereof; about 0.0098% 0.001% anhydrous citric acid; about 0.279%±0.028% sodium citrate dihydrate; about 0.001%±0.0001% disodium EDTA; about 0.01%±0.001% tyloxapol; about 0.50%±0.05% NaCl; wherein the pH of the composition is adjusted using NaOH or HCl to be about 6.2 to about 6.8, and the osmolality is 170-210 mOsm/kg. In some embodiments, the composition is a sterile aqueous composition comprising, consisting or consisting essentially of about 0.005%±0.0005% of a polypeptide, such as Lacripep™ (SEQ ID NO. 1) or the other peptides identified herein, or a pharmaceutically acceptable salt thereof; about 0.0098%±0.001% anhydrous citric acid; about 0.279%±0.028% sodium citrate dihydrate; about 0.001%±0.0001% disodium EDTA; about 0.05%±0.005% tyloxapol; about 0.50%±0.05% NaCl; wherein the pH of the composition is adjusted using NaOH or HCl to be about 6.2 to about 6.8, and the osmolality is 170-210 mOsm/kg. In an embodiment, the pH is about 6.3 to about 6.5 and the osmolality is 180-200 mOsm/kg. In an embodiment, the composition consists of only the listed ingredients, and does not contain any additional active ingredients, excipients (e.g., viscosity building agents, buffering agents, chelating agents, stabilizing agents, preservatives, surfactants, and tonicity agents), carriers or diluents.

In some embodiments, the composition is a sterile aqueous composition comprising, consisting or consisting essentially of about 0.001%±0.0001% of a polypeptide, such as Lacripep™ (SEQ ID NO. 1) or the other peptides identified herein, or a pharmaceutically acceptable salt thereof; about 0.0098%±0.001% anhydrous citric acid; about 0.279%±0.028% sodium citrate dihydrate; about 0.001%±0.0001% disodium EDTA; about 0.001%±0.0001% tyloxapol; about 0.50% 0.05% NaCl; wherein the pH of the composition is adjusted using NaOH or HCl to be about 6.2 to about 6.8, and the osmolality is 170-210 mOsm/kg. In some embodiments, the composition is a sterile aqueous composition comprising, consisting or consisting essentially of about 0.001%±0.0001% of a polypeptide, such as Lacripep™ (SEQ ID NO. 1) or the other peptides identified herein, or a pharmaceutically acceptable salt thereof; about 0.0098%±0.001% anhydrous citric acid; about 0.279%±0.028% sodium citrate dihydrate; about 0.001%±0.0001% disodium EDTA; about 0.01%±0.001% tyloxapol; about 0.50%±0.05% NaCl; wherein the pH of the composition is adjusted using NaOH or HCl to be about 6.2 to about 6.8, and the osmolality is 170-210 mOsm/kg. In some embodiments, the composition is a sterile aqueous composition comprising, consisting or consisting essentially of about 0.001%±0.0001% of a polypeptide, such as Lacripep™ (SEQ ID NO. 1) or the other peptides identified herein, or a pharmaceutically acceptable salt thereof; about 0.0098% 0.001% anhydrous citric acid; about 0.279%±0.028% sodium citrate dihydrate; about 0.001%±0.0001% disodium EDTA; about 0.05%±0.005% tyloxapol; about 0.50%±0.05% NaCl; wherein the pH of the composition is adjusted using NaOH or HCl to be about 6.2 to about 6.8, and the osmolality is 170-210 mOsm/kg. In an embodiment, the pH is about 6.3 to about 6.5 and the osmolality is 180-200 mOsm/kg. In an embodiment, the composition consists of only the listed ingredients, and does not contain any additional active ingredients, excipients (e.g., viscosity building agents, buffering agents, chelating agents, stabilizing agents, preservatives, surfactants, and tonicity agents), carriers or diluents.

In some embodiments, the composition is a sterile aqueous composition comprising, consisting or consisting essentially of about 0.00025%±0.000025% of a polypeptide, such as Lacripep™ (SEQ ID NO. 1) or the other peptides identified herein, or a pharmaceutically acceptable salt thereof; about 0.0098% 0.001% anhydrous citric acid; about 0.279%±0.028% sodium citrate dihydrate; about 0.001%±0.0001% disodium EDTA; about 0.001%±0.0001% tyloxapol; about 0.50%±0.05% NaCl; wherein the pH of the composition is adjusted using NaOH or HCl to be about 6.2 to about 6.8, and the osmolality is 170-210 mOsm/kg. In some embodiments, the composition is a sterile aqueous composition comprising, consisting or consisting essentially of about 0.00025%±0.000025% of a polypeptide, such as Lacripep™ (SEQ ID NO. 1) or the other peptides identified herein, or a pharmaceutically acceptable salt thereof; about 0.0098%±0.001% anhydrous citric acid; about 0.279%±0.028% sodium citrate dihydrate; about 0.001%±0.0001% disodium EDTA; about 0.01%±0.001% tyloxapol, about 0.50%±0.05% NaCl; wherein the pH of the composition is adjusted using NaOH or HCl to be about 6.2 to about 6.8, and the osmolality is 170-210 mOsm/kg. In some embodiments, the composition is a sterile aqueous composition comprising, consisting or consisting essentially of about 0.00025%±0.000025% of a polypeptide, such as Lacripep™ (SEQ ID NO. 1) or the other peptides identified herein, or a pharmaceutically acceptable salt thereof; about 0.0098%±0.001% anhydrous citric acid; about 0.279%±0.028% sodium citrate dihydrate; about 0.001%±0.0001% disodium EDTA; about 0.05%±0.005% tyloxapol; about 0.50%±0.05% NaCl; wherein the pH of the composition is adjusted using NaOH or HCl to be about 6.2 to about 6.8, and the osmolality is 170-210 mOsm/kg. In an embodiment, the pH is about 6.3 to about 6.5 and the osmolality is 180-200 mOsm/kg. In an embodiment, the composition consists of only the listed ingredients, and does not contain any additional active ingredients, excipients (e.g., viscosity building agents, buffering agents, chelating agents, stabilizing agents, preservatives, surfactants, and tonicity agents), carriers or diluents.

In some embodiments, including but not limited to the sterile compositions above, the polypeptide is Lacripep™, having SEQ ID NO: 1, or a pharmaceutically acceptable salt thereof. In some embodiments the polypeptide is a polypeptide having SEQ ID NO: 2, or a pharmaceutically acceptable salt thereof. In some embodiments the polypeptide is a polypeptide having a sequence selected from the group of SEQ ID NOs:3-9, or a pharmaceutically acceptable salt or fragment or fragments thereof.

In some embodiments, including but not limited to the sterile compositions above, the pH of the composition is about 6.5 to about 6.6. In some embodiments, including but not limited to the sterile compositions above, the pH of the composition is about 6.3 to about 6.5. In some embodiments, including but not limited to the sterile compositions above, the pH of the composition is about 6.5.

In some embodiments, including but not limited to the sterile compositions above, the osmolality of the composition is about 270 to about 210 mOsm/kg. In some embodiments, including but not limited to the sterile compositions above, the osmolality of the composition is about 180 to about 200 mOsm/kg. In some embodiments, the osmolality of the composition is about 190 mOsm/kg.

In an embodiment, including but not limited to the sterile compositions above, the composition consists of only the listed ingredients, and does not contain any additional active ingredients, excipients (e.g., viscosity building agents, buffering agents, chelating agents, stabilizing agents, preservatives, surfactants, and tonicity agents), carriers or diluents. In some embodiments, the amounts of any one or more of the listed ingredients is provided in an amount that is ±5%, and/or ±1% of the listed amount.

In some embodiments, the compositions disclosed herein are prepared as a solution, gel or ointment. Gels or ointments are advantageous in providing the composition in contact with the eye for a longer period of time than a solution or provide other benefits. Therefore, in some embodiments, a gel or ointment is useful when applying the composition to the subject when the subject will be sleeping, or when the subject's eyes will be closed for an extended period of time (e.g., 1, 2, 3, 4, 5 or more hours). Gels or ointments may be used at other times based on user preference.

Non-limiting exemplary compositions, which can be used in the methods and kits disclosed herein, include the following compositions in Tables A, B and C below, as well as in Tables 1.1, 1.2 and 1.3 in the Examples.

TABLE A

| Component | | | |
|---|---|---|---|
| (% w/v) | A | B | C |
| Lacripep ™ | 0.005 | 0.001 | 0.00025 |
| (SEQ ID NO. | | | |
| 1) | | | |
| Citric Acid | 0.0098 | 0.0098 | 0.0098 |
| (Anhydrous) | | | |
| Sodium | 0.279 | 0.279 | 0.279 |
| Citrate | | | |
| (Dihydrate) | | | |
| EDTA | 0.001 | 0.001 | 0.001 |
| Disodium | | | |
| Tyloxapol | 0.001 | 0.001 | 0.001 |
| NaCl | 0.5 | 0.5 | 0.5 |
| 10% NaOH | Adjust pH | Adjust pH | Adjust pH |
| (aq) or 10% | to 6.5 ± 0.3 | to 6.5 ± 0.3 | to 6.5 ± 0.3 |
| HCl (aq) | | | |
| Sterile | qs ad 100 | qs ad 100 | qs ad 100 |
| Purified Water | | | |

TABLE B

| Component | | | |
|---|---|---|---|
| (% w/v) | D | E | F |
| Lacripep ™ | 0.005 | 0.001 | 0.00025 |
| (SEQ ID NO. | | | |
| 1) | | | |
| Citric Acid | 0.0098 | 0.0098 | 0.0098 |
| (Anhydrous) | | | |
| Sodium | 0.279 | 0.279 | 0.279 |
| Citrate | | | |
| (Dihydrate) | | | |
| EDTA | 0.001 | 0.001 | 0.001 |
| Disodium | | | |
| Tyloxapol | 0.01 | 0.01 | 0.01 |
| NaCl | 0.5 | 0.5 | 0.5 |
| 10% NaOH | Adjust pH | Adjust pH | Adjust pH |
| (aq) or 10% | to 6.5 ± 0.3 | to 6.5 ± 0.3 | to 6.5 ± 0.3 |
| HCl (aq) | | | |
| Sterile | qs ad 100 | qs ad 100 | qs ad 100 |
| Purified Water | | | |

TABLE C

| Component | | | |
|---|---|---|---|
| (% w/v) | G | H | I |
| Lacripep ™ | 0.005 | 0.001 | 0.00025 |
| (SEQ ID NO. | | | |
| 1) | | | |
| Citric Acid | 0.0098 | 0.0098 | 0.0098 |
| (Anhydrous) | | | |
| Sodium | 0.279 | 0.279 | 0.279 |
| Citrate | | | |
| (Dihydrate) | | | |
| EDTA | 0.001 | 0.001 | 0.001 |
| Disodium | | | |
| Tyloxapol | 0.05 | 0.05 | 0.05 |
| NaCl | 0.5 | 0.5 | 0.5 |
| 10% NaOH | Adjust pH | Adjust pH | Adjust pH |
| (aq) or 10% | to 6.5 ± 0.3 | to 6.5 ± 0.3 | to 6.5 ± 0.3 |
| HCl (ag) | | | |
| Sterile | qs ad 100 | qs ad 100 | qs ad 100 |
| Purified Water | | | |

Embodiments of the compositions of Tables A, and/or B, and/or C (and Tables 1.1 and/or 1.2 and/or 1.3) also include compositions with a pH of 6.3 to 6.5. Embodiments of the compositions of Tables A, and/or B, and/or C (and Tables 1.1 and/or 1.2 and/or 1.3) also include compositions with the amounts of the disclosed ingredients in a range of ±1% of the disclosed amount, in a range of ±2% of the disclosed amount, in a range of ±3% of the disclosed amount, in a range of ±4% of the disclosed amount, or in a range of ±5% of the disclosed amount. In some embodiments, compositions of Tables A, and/or B, and/or C (and Tables 1.1 and/or 1.2 and/or 1.3) maintain at least about 99.0%/o, 99.9%, 99.95%, or 99.99% of the Lacripep™ polypeptide of SEQ ID NO: 1 in its initial, undegraded form in the composition after storage of the composition for at least 1 or 2 weeks, 1, 2, 3, 4 or 5 months at −20±5° C., 5±3° C. or 25±2° C. and 25±5% relative humidity. In some embodiments, compositions of Tables A, and/or B, and/or C (and Tables 1.1 and/or 1.2 and/or 1.3) maintains at least about 80% or 90% of the Lacripep™ polypeptide of SEQ ID NO: 1 in its initial, undegraded form in the composition after storage of the composition for at least 12 months at −20±5° C. or 5±3° C.

### Other Therapeutic Ingredients

In some embodiments the compositions include one or more additional therapeutic agents in addition to the polypeptides disclosed herein. These therapeutic agents can include substances known to those skilled in the art for the treatment of dry eye and related syndromes and conditions, including general dry eye disease. The additional therapeutic ingredients can treat the disease, syndrome or condition, or can relieve symptoms associated with the disease, syndrome or condition. A non-exhaustive list of additional therapeutic agents includes: cholinergics (e.g., pilocarpine, cevimeline), Cyclosporine, Lifitegrast, Dexamethasone (or other cortico-steroids such as prednisolone), Hyaluronic acid (and its derivatives) with or without chondroitin sulfate, Cyclokat, SI-614, skQ1, Cis-UCA, CycloASol, RGN-259, Diquafosol, Anakinra, Tofacitinib, EBI-005, EGP-437, KP-121, MIM-D3, OTX-DP, rebamipide (OPC-12759), and RU-101. In some embodiments, the additional therapeutic agent is Xiidra (lifitegrast, SAR-1118). In some embodiments, the one or more additional therapeutic agents are provided as a salt of the polypeptide. Artificial tears and other lubricants that contain one or more of carboxymethyl cellulose, polyvinyl alcohol, hydroxypropyl methylcellulose (a.k.a. HPMC or hypromellose), hydroxypropyl cellulose, ethylene glycol polymers, and hyaluronic acid (a.k.a. hyaluronan, HA), and tear ointments such as white petrolatum, mineral oil, and similar lubricants can also be included in the compositions. These additional therapeutic agents can be included in known therapeutic amounts, or sub-therapeutic amounts.

### Containers and Kits

In some embodiments, the composition is provided in a kit comprising one or more multi-use containers. In some embodiments, the multi-use container comprises a protective cap and a liquid storage bottle, wherein the cap is connected to the bottle via a flexible connector. A blocking plug is arranged in the middle of the top surface of the protective cap. A conical, or other suitable shape, liquid outlet is arranged in the middle of the bottle cover and is tightly matched with the blocking plug of the protective cap. Thus, the sterile composition may be placed into the container for multiple uses.

In some embodiments, the amount of the composition in the container is, or is about: 0.1-0.5, 0.5-1.0, 1-2, 2-5, 5-10, 10-20, 20-30, or 30-60 mL or ranges in between. Containers may be bottles, tubes, vials or other suitable containers. Multi-use containers may be accompanied by instructions to use for a 12 hour, 24 hour, 2-7 day cycle, one month cycle or until a stated expiration date. A single-use container may be suitable for use in one eye or both eyes for a single application cycle.

In some embodiments, the composition is provided in a in a kit comprising a single-use container. In some embodiments, the composition is provided in a kit comprising a plurality of single-use containers. In some embodiments, the single-use container comprises a vessel for holding liquid, a removable seal top for sealing the vessel, and, optionally, a neck portion interconnecting the vessel and the seal top. Kits comprises multiple single-use containers along with instructions to use are provided in several embodiments.

In some embodiments, the container comprises a pharmaceutically inert material. In some embodiments, the container comprises glass, polyvinyl chloride, polypropylene, polyethylene terephthalate, polyethylene terephthalate, polyethylene terephthalate G, high-density polyethylene, low-density polyethylene, polybutylene terephthalate, polyurethane, polyethylene vinyl acetate, silicone, acrylonitrile butadiene styrene, polytetrafluoroethylene, polycarbonate, polystyrene, polymethylmethacrylate, polysulfone, polyvinylidene chloride, or combinations thereof.

In some embodiments, the container comprises polyvinyl chloride, polypropylene, low-density polyethylene, polyurethane, polyethylene vinyl acetate, silicone, or combinations thereof.

In some embodiments, the amount of composition in the container is, or is about, 0.02 mL; 0.05 mL to 1 mL; 0.1 mL to 0.95 mL; 0.15 mL to 0.8 mL; 0.2 mL to 0.85 mL; 0.25 mL to 0.8 mL; 0.3 mL to 0.75 mL; 0.35 mL to 0.7 mL; 0.4 mL to 0.65 mL; 0.45 mL to 0.6 mL; 0.5 mL to 0.55 mL; or any amount in between.

In some embodiments, the amount of composition in the container is, or is about, 0.02 mL; 0.025 mL; 0.030 mL; 0.035 mL; 0.040 mL; 0.045 mL; 0.050 mL; 0.055 mL; 0.060 mL; 0.065 mL; 0.070 mL; 0.075 mL; 0.1 mL; 0.15 mL; 0.2 mL; 0.25 mL; 0.3 mL; 0.35 mL; 0.4 mL; 0.45 mL; 0.5 mL; 0.55 mL; 0.6 mL; 0.65 mL; 0.7 mL; 0.75 mL; 0.8 mL; 0.85 mL; 0.9 mL; 0.95 mL; or 1 mL of the composition, or an amount that is within a range defined by any two of the preceding values.

### Ophthalmic and Other Administration

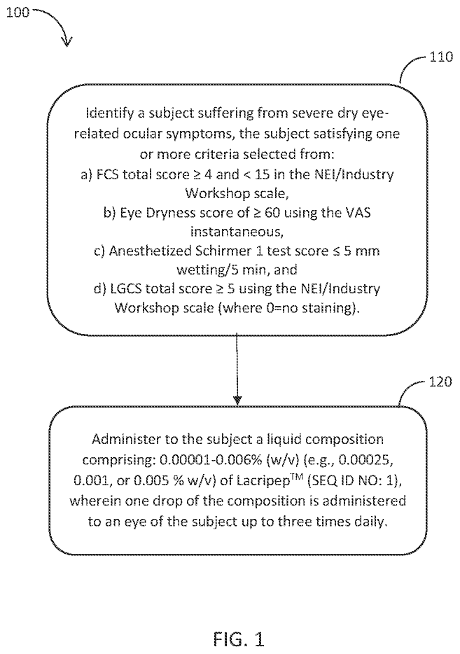

Provided herein are methods for treating a moderate/severe dry eye-related ocular symptom by administering a polypeptide as described herein to the eye of a subject identified as suffering from moderate/severe dry eye and in need thereof. In some embodiments, the subject is identified as suffering from severe dry eye. In some embodiments, the subject does not have Sjögren's Syndrome (Primary or Secondary). With reference to FIG. 1, a method of treating a moderate/severe dry eye-related ocular symptom in a subject (e.g., a subject having general dry eye) is provided. The method 100 can include, at block 110, identifying a subject suffering from one or more moderate/severe dry eye-related ocular symptoms (e.g., a subject having general dry eye, and optionally not having Sjögren's Syndrome (Primary or Secondary)) who satisfies one or more (e.g., 1, 2, 3, or 4) of the listed criteria:
a) FCS total score ≥4 and <15 in the NEI/Industry Workshop scale,
b) Eye Dryness score of ≥60 using the VAS instantaneous.
c) Anesthetized Schirmer 1 test score ≤5 mm wetting/5 min, and
d) LGCS total score ≥5 using the NEL/Industry Workshop scale (where 0=no staining).

In some embodiments, a subject suffering from moderate/severe dry eye satisfies all four of the above criteria in at least one eye. In some embodiments, the subject is identified as suffering from severe dry eye by having a SANDE score 60 (not shown).

At block 120, the method includes administering to the subject a composition of the present disclosure, e.g., a liquid composition that includes an amount of a polypeptide of the present disclosure (e.g., Lacripep™ (SEQ ID NO: 1), or a pharmaceutically acceptable salt thereof, for example a composition in Table A, B or C, or 1.1, 1.2, or 1.3.

In some embodiments, the subject does not have moderate/severe or severe dry eye and/or dry eye symptoms in at least one eye when the subject meets at least one of the following criteria in the at least one eye:
a) FCS total score <4 or ≥15 in the NEI/Industry Workshop scale,
b) Eye Dryness score of <60 using the VAS instantaneous,
c) Anesthetized Schirmer 1 test score >5 mm wetting/5 min, or
d) LGCS total score <5 using the NEI/Industry Workshop scale (where 0=no staining).

In some embodiments, the subject does not have moderate/severe or severe dry eye and/or dry eye symptoms in at least one eye when the FCS total score is <4 in the NEI/Industry Workshop scale in the at least one eye. In some embodiments, the subject does not have moderate/severe or severe dry eye and/or dry eye symptoms in at least one eye when the Eye Dryness score is <60 using the VAS instantaneous in the at least one eye. In some embodiments, the subject does not have moderate/severe or severe dry eye and/or dry eye symptoms in at least one eye when the Anesthetized Schirmer 1 test score is >5 mm wetting/5 min in the at least one eye. In some embodiments, the subject does not have moderate/severe or severe dry eye and/or dry eye symptoms in at least one eye when the LGCS total score is <5 using the NEI/Industry Workshop scale (where 0=no staining) in the at least one eye.

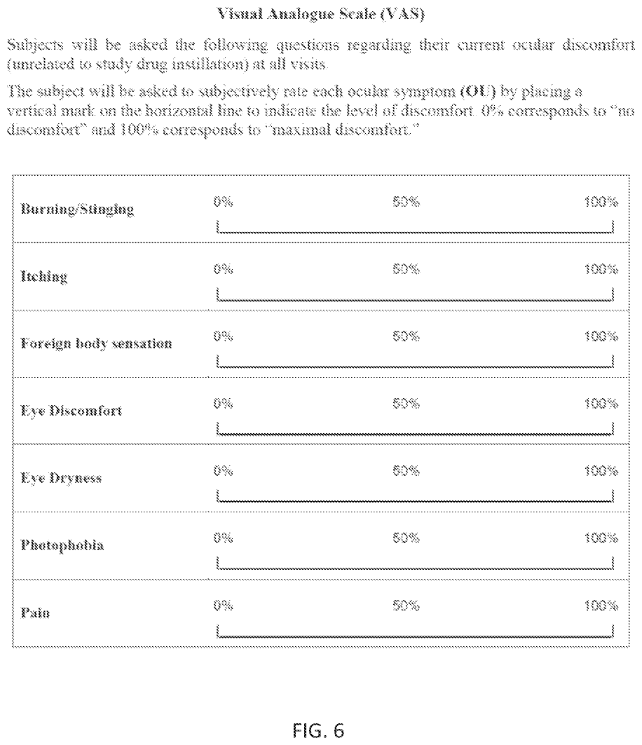

The subject suffering from one or more moderate/severe dry eye-related ocular symptoms can be identified using any suitable option. In some embodiments, a subject is identified as suffering from moderate/severe dry eye-related ocular symptoms based at least on the subject's assessment of one or more dry eye-related ocular symptoms, such as, without limitation, pain, stinging, burning, or itching sensations. In some embodiments, a subject is identified as suffering from moderate/severe dry eye-related ocular symptoms based at least on the subject's assessment of ocular stinging and/or burning. In some embodiments, the assessment of symptoms is an instantaneous assessment of symptoms by the subject. In some embodiments, the symptoms are rated on a visual analogue scale (VAS), e.g., on a scale of 0-100 mm, 0 mm being minimum intensity, and 100 mm being maximum intensity, to provide an Eye Dryness Score (EDS) (e.g., on a scale of 0-100 mm, 0 mm being minimum score, and 100 mm being maximum score). The EDS can be obtained by the subject's assessment of symptoms on a questionnaire, for example, as shown in FIG. 6. Using VAS, the subject's reported assessment of their symptoms on the linear dimensions of this scale is measured in millimeters (e.g., taking the left end of each line as 0) to provide the EDS. (See Schaunberg D, et al. Development and Validation of a Short Global Dry Eye Symptom Index. The Ocular Surface. January 2007, Vol 5; 1; 50-57, which is hereby incorporated by reference in its entirety). The EDS score is the value for the “Eye Dryness” line on a VAS questionnaire, e.g., the questionnaire in FIG. 6.

In some embodiments, a subject is identified as suffering from moderate/severe dry eye-related ocular symptoms at least when the subject has an EDS at or above a predetermined EDS threshold. In some embodiments, the EDS threshold is determined based on the distribution of EDS in a cohort of patients known to have moderate/severe dry eye disease. In some embodiments, the EDS threshold is determined based on the distribution of EDS in a cohort of subjects known not to have moderate/severe dry eye disease. In some embodiments, the EDS threshold is 60, 65, 70, 75, 80, 85, 90 or more, or a value in a range defined by any two of the preceding values (e.g., 60-90, 60-80, 60-70, or 60-100). In some embodiments, the one or more dry eye-related ocular symptoms includes an EDS at or above 60. In some embodiments, the subject has general dry eye disease. In some embodiments, the subject does not have Sjögren's Syndrome (Primary or Secondary).

In some embodiments, the method includes at least using a subject's response to one or more questionnaires that are designed to measure, for example and without limitation, one or more of: ocular surface discomfort or vision symptoms associated with dry eye disease; the impact of dry eye disease on everyday function; and health-related quality of life, can be used, in lieu of or in addition to the subject's instantaneous assessment of one or more dry eye-related ocular symptoms on a VAS scale as discussed above, to identify a subject as suffering from dry eye-related ocular symptoms. In some embodiments, the questionnaire includes, without limitation, one or more of 1) Ocular Surface Disease Index (OSDI); 2) Impact of Dry Eye on Everyday Life (IDEEL); 3) National Eye Institute-Visual Function Questionnaire (NEIVFQ); 4) Symptom Assessment in Dry Eye (SANDE); 5) Dry Eye-Related Quality-of-Life Score Questionnaire (DEQS); 6) McMonnies Dry Eye Questionnaire; 7) Women's Health Study Questionnaire: 8) Dry Eye Questionnaire (DEQ); 9) North Carolina Dry Eye Management Scale (UNC DEMS); 10) Subjective Evaluation of Symptom of Dryness (SESoD); 11) Standard Patient Evaluation of Eye Dryness (SPEED); 12) Dry Eye Epidemiology Project Questionnaire (DEEP); 13) Canada Dry Eye Epidemiology Study (CANDEES); 14) Salisbury eye evaluation; 15) Melbourne visual impairment project; 16) Bjerrum questionnaire; 17) Japanese dry eye awareness study. Suitable, non-limiting examples of questionnaires are summarized in Stapleton et al. TFOS DEWS II Epidemiology Report. Ocul Surf. 2017 July; 15(3):334-365, and Wolffsohn et al. TFOS DEWS II Diagnostic Methodology report. Ocul Surf. 2017 Jul; 15(3):539-574, each of which is incorporated herein by reference in its entirety. In some embodiments, a subject is identified as suffering from moderate/severe dry eye-related ocular symptoms at least when the measurement of symptoms by any suitable questionnaire, e.g., as discussed above, is at or above a threshold measurement that corresponds to the EDS threshold for the subject's instantaneous assessment of one or more dry eye-related ocular symptoms in the VAS scale. In some embodiments, the subject does not have Sjögren's Syndrome (Primary or Secondary).

In some embodiments, a subject suffering from one or more moderate/severe dry eye-related ocular symptoms is identified at least by one or more ocular measurements, e.g., one or more ocular symptoms that are not measured based on reporting by the subject. Any suitable ocular measurements can be used to identify the subject as suffering from one or more dry eye-related ocular symptoms. In some embodiments, symptoms of dry eye are determined based at least on measurement of, without limitation, one or more of tear film stability, tear secretion, tear volume, tear film composition, damage to ocular surface, inflammation of the ocular surface, and eye lid function. In some embodiments, symptoms of dry eye are determined at least by, without limitation, one or more of elevated corneal staining, elevated conjunctival staining, decreased tear breakup time, and decreased Schirmer's tear test score (with or without anesthesia). Suitable, non-limiting examples of ocular measurements are summarized in Stapleton et al. TFOS DEWS II Epidemiology Report. Ocul Surf. 2017 Jul; 15(3):334-365, and Wolffsohn et al. TFOS DEWS II Diagnostic Methodology report. Ocul Surf. 2017 July; 15(3):539-574, each of which is incorporated herein by reference in its entirety. In some embodiments, the subject does not have Sjögren's Syndrome (Primary or Secondary).

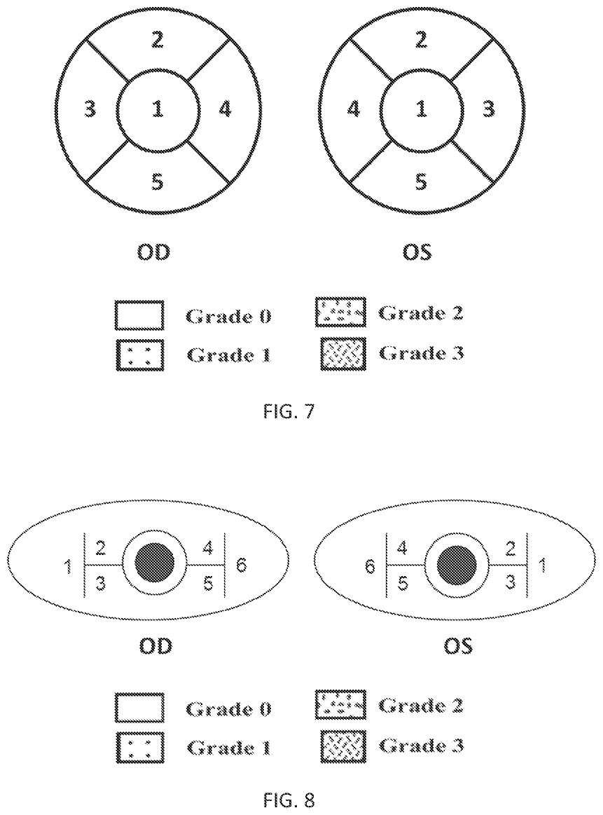

In some embodiments, symptoms of moderate/severe dry eye are determined at least by, without limitation, one or more of the Schirmer test (e.g., anesthetized Schirmer test), Lissamine green conjunctival staining (LGCS), and Fluorescein corneal staining (FCS). “Fluorescein corneal staining”, “FCS”, “corneal fluorescein staining”, and “CFS” are used interchangeably herein. In some embodiments, the method includes measuring ocular surface staining, e.g., FCS or LGCS, to determine whether a subject has one or more dry eye-related ocular symptoms. In some embodiments, ocular surface staining is scored using the National Eye Institute (NEI)/Industry Workshop scale. In some embodiments, the FCS in the NEI/Industry Workshop scale is determined by a trained practitioner (e.g., trained ophthalmologist) scoring the level of CFS within a grid that divides the corneal area into five sections (e.g., as shown in FIG. 7), each of which is assigned a score (or grade) from zero and 3 depending on the amount and distribution of staining. Score/grade 0 indicates no staining in a section. The total FCS score can range from 0/15 (indicating absence of corneal epitheliopathy) to 15/15 (indicating severe epitheliopathy) in the NEI/Industry Workshop scale. In some embodiments, the LGCS in the NEI/Industry Workshop scale is determined by a trained practitioner (e.g., trained ophthalmologist) scoring the level of LGCS within a grid that divides the conjunctiva area into six sections (superior paralimbal, inferior paralimbal, peripheral area both nasally & temporally) (e.g., as shown in FIG. 8), each of which is assigned a score (or grade) from zero and 3 depending on the amount and distribution of staining. Score/grade 0 indicates no staining in a section. The total LGCS score can range from 0/18 to 18/18 in the NEI/Industry Workshop scale.

Ocular surface staining can be scored using any suitable equivalent option to the NEI/industry Workshop scale detailed above. In some embodiments, ocular surface staining is scored according to one or more of the following grading scales: van Bijsterveld system, the Collaborative Longitudinal Evaluation of Keratoconus (CLEK) schema, the Oxford Scheme, the area-density combination index, and the Sjögren's International Collaborative Clinical Alliance ocular staining score. Suitable, non-limiting examples of scoring ocular surface staining is summarized in Wolffsohn et al. TFOS DEWS 1 Diagnostic Methodology report. Ocul Surf. 2017 July; 15(3):539-574, which is hereby incorporated by reference in its entirety.

In some embodiments, the subject is identified as suffering from moderate/severe dry eye-related ocular symptoms at least when the subject's FCS total score is above a FCS threshold. In some embodiments, the one or more dry eye-related ocular symptoms includes a FCS total score of >about 4 and <about 15 in the NEI/Industry Workshop scale. In some embodiments, the FCS threshold is 4, 5, 6, 7, 8, 9, 10, 11, 12, 13, 14, or 15, in the NEI/Industry Workshop scale, or a value in a range defined by any two of the preceding values (e.g., 4-14, 6-12, 4-10, 6-8, 6-14, 8-14, 8-12 or 4-6), where 0 indicates no staining. In some embodiments, the subject is identified as suffering from dry eye-related ocular symptoms at least when the subject's FCS total score as determined using an alternative scoring option is at or above a threshold that would correspond to the threshold as defined using the NEI/Industry Workshop scale.

In some embodiments, the subject is identified as suffering from moderate/severe dry eye-related ocular symptoms at least when the subject's Lissamine green conjunctival staining (LGCS) score is above a LGCS threshold. In some embodiments, the one or more dry eye-related ocular symptoms includes a LGCS score >5, in the National Eye Institute (NET)/Industry Workshop scale, where 0 indicates no staining. In some embodiments, the LGCS threshold is at least 5, 6, 7, 8, 9, 10, 11, 12, 13, 14, or higher, in the NEI/Industry Workshop scale, or a value in a range defined by any two of the preceding values (e.g., 5-14, 5-12, 8-14, 5-8, 10-14, or 5-6), where 0 indicates no staining. In some embodiments, the subject is identified as suffering from dry eye-related ocular symptoms at least when the subject's LGCS score as determined using an alternative scoring option is at or above a threshold that would correspond to the threshold as defined using the NEI/Industry Workshop scale.

In some embodiments, the subject is identified as suffering from moderate/severe dry eye-related ocular symptoms at least based on a measurement of tear secretion. In some embodiments, tear secretion is measured using a Schirmer test, or a variant thereof. The Schirmer test can be any suitable form of the Schirmer test. In some embodiments, the one or more dry eye-related ocular symptoms includes an anesthetized Schirmer test score of ≤5 mm wetting/5 min. In some embodiments, the Schirmer test, without limitation, is anesthetized Schirmer 1, Schirmer 2, Schirmer 3, a thread method, or a 1-minute Schirmer test. In some embodiments, the one or more dry eye-related ocular symptoms includes a Schirmer test score that is below a Schirmer test threshold, e.g., an anesthetized Schirmer 1 test score that is below an anesthetized Schirmer 1 test threshold. A suitable Schirmer test and variants thereof, as well as corresponding threshold values, are summarized in Savini et al. The challenge of dry eye diagnosis. Clin Ophthalmol. 2008 March; 2(1):31-55, which is incorporated by reference in its entirety. In some embodiments, the Schirmer test is an anesthetized Schirmer 1 test. In some embodiments, the one or more dry eye-related ocular symptoms includes an anesthetized Schirmer test score of ≤5 mm wetting/5 min. In some embodiments, any one of the Schirmer test variants (including anesthetized Schirmer 1) can be used to identify a subject as suffering from one or more moderate/severe dry eye-related ocular symptoms.

In some embodiments, the subject is identified as suffering from severe dry, rather than moderate/severe dry eye, by having a SANDE score ≥60.

In some embodiments, the subject has a history of moderate/severe dry eye-related ocular symptoms. In some embodiments, the method includes determining that the subject has a history of one or more moderate/severe dry eye-related ocular symptoms. In some embodiments, the subject has experienced or has been diagnosed with one or more prior moderate/severe dry eye-related ocular symptoms at least 1, 2, 3, 4, 5, 6, 7, 8, 9, 10, 11, 12, 16, 20, 24, 30, 36 months or more, or by a length of time in a range defined by any two of the preceding values (e.g., 1-36, 1-24, 1-12, 3-30, 3-24, 6-12, 6-24 months) before being identified as suffering from one or more dry eye-related ocular symptoms. In some embodiments, the subject the one or more moderate/severe dry eye-related ocular symptoms comprises the subject's use of ocular wetting agents (e.g., artificial tears) within the last 6, 5, 4, 3, 2, or 1 months, or within a length of time in a range defined by any two of the preceding values (e.g., 1-6, 2-6, 3-5 months).

In some embodiments, the subject is at least 18 years old. In some embodiments, the subject is 18-25, 25-40, 40-55, 55-60, 60-65, 65-70, 70-75, 75-80, 80-85 years old, or older.

In some embodiments, a subject meeting one or more of the following are excluded:
1. Subjects with any active infectious ocular condition.
2. Subjects who are monocular or have a BCVA, using corrective lenses if necessary, of +1.0 logMAR or worse as assessed by Early Treatment Diabetic Retinopathy Study (ETDRS).
3. Subjects with ocular inflammatory conditions (e.g., conjunctivitis, keratitis, anterior blepharitis, etc.) not related to dry eye syndrome.
4. Subjects with clinical evidence of cicatricial ocular surface disease, such as cicatricial ocular pemphigoid or Stevens Johnson syndrome.
5. Subjects who cannot suspend the use of any topical eye medications (including topical cyclosporine) other than the composition during the run-in and the treatment phase.
6. Subjects who have used Restasis® (topical ophthalmic cyclosporine) within 60 days prior to visit 1.
7. Subjects who have used Xiidra® (topical ophthalmic lifitegrast) within 60 days prior to Visit 1.
8. Subjects who in the treated eye have fluorescein corneal staining (FCS) Total Score=15 or a Score=3 in the superior region per the NEI/Industry Workshop scale or subjects who have FCS with diffuse confluent staining, filaments or epithelial defects.
9. Subjects who have active or have had an outbreak of herpetic keratitis within 365 days before identification or subjects who are on chronic oral antivirals for herpetic disease.
10. Subjects who cannot suspend the use of and abstain from contact lens use from during administration.
11. Subjects who have used or anticipate use of amiodarone.
12. Subjects who within 30 days prior to identification alter the dose or anticipate alterations to the dose of the following: tetracyclines, Omega 3's or 6's.
13. Subjects who within 60 days prior to identification and for the duration of the treatment alter the dose or anticipate alterations to the dose of the following: anticholinergics, antidepressants, oral contraceptives, isotretinoin, oral systemic corticosteroids, oral systemic immunosuppressive agents.
14. Subjects who within 30 days prior to identification and for the duration of the treatment use topical ocular antihistamines, ocular, inhaled or intranasal corticosteroids, topical or oral mast cell stabilizers, oral antihistamines, topical or nasal vasoconstrictors, topical ocular NSAIDs, topical ocular antibiotics.
15. Subjects who in the treated eye have had cauterization of the punctum or alterations to (insertion or removal) punctal plug(s) within the past 90 days prior to identification. Note: If a punctal plug in place at Randomization/Baseline and it is dislodged, the plug should be replaced as soon as possible.
16. Subjects who, in the treated eye, have had corneal refractive surgery (LASIK, PRK, RK).
17. Subjects who in the treated eye, have a history of any operative procedure on the ocular surface or eyelids within 365 days prior to identification or with a history of intraocular surgery within 90 days prior to identification.
18. Subjects who are pregnant or suspected to be pregnant and subjects who are breastfeeding or intend to breastfeed. Female subjects of childbearing potential are required to have a negative urine pregnancy test at screening, and must agree to use an acceptable method of contraception from the time of signing informed consent until the end of treatment. Medically acceptable contraception methods include intrauterine device; barrier methods such as diaphragm, condom, cap or sponge, used with a spermicide; or hormonal contraception.
19. Subjects with any physical or mental impairment that would preclude participation and the ability to give informed consent.
20. Subjects who have participated in a device or Investigational drug study or clinical trial within 30 days of identification. Participation in another during treatment is excluded for the duration of the treatment.
   In some embodiments the subject does not have Sjögren's Syndrome (Primary or Secondary).

In some embodiments, the composition is administered topically to the eye. In some embodiments, the composition is administered to an individual suffering from (e.g., identified as suffering from) any form of moderate/severe dry eye, for the treatment thereof. In some embodiments the subject does not have Sjögren's Syndrome (Primary or Secondary). The compositions described herein can be provided as liquids (solutions, gels, ointments etc.) or in other suitable forms, such as powders or on patches, tabs, etc. In some embodiments, the compositions described herein are used to achieve one or more of the following: restore basal tearing, general mucosal and ocular surface wetness; restore ocular surface and mucosal homeostasis, rapidly but transiently promote autophagy to eliminate pressure, stress or degenerative disease throughout the eye and in other organs; reduce inflammation, promote wound healing (such as corneal post refractive surgery or oral wound healing), stabilize the tear lipid layer and suppress bacterial infection.

In some embodiments, administration topically to the eye comprises administering one or more drops of the composition to the surface of the eye. For example, in some embodiments, a user is instructed to apply to the eye surface, and not to a contact lens). In other embodiments, the drops (or other application) is suitable for administration while wearing contact lenses. In some embodiments, the composition is administered from the container as a single dose delivered as a single drop to each eye. In some embodiments, the drop is about 0.020 mL to about 0.050 mL, or any volume in between. In some embodiments, the drop is about 0.035 mL.

The composition can be administered to the subject at any suitable dosing frequency and volume. In some embodiments, the composition is administered as one or more drops (e.g., 1, 2, 3, 4, 5 or more drops) to the eye (e.g., the eye surface) of the subject. In some embodiments, one drop of the composition is administered to the eye of the subject per administration. In some embodiments, one drop of the composition includes a volume of the composition of about 0.005 ml, about 0.01 ml, about 0.02 ml, about 0.03 ml, 0.04 ml, about 0.05 ml, about 0.06 ml, about 0.07 ml, about 0.08 ml, about 0.09 ml, about 0.1 ml, about 0.2 ml, about 0.5 ml or more, or a volume in a range defined by any two of the preceding values (e.g., 0.005-0.5 ml, 0.01-0.1 ml, 0.02-0.08 ml, 0.03-0.07 ml, etc.). In some embodiments, one or more drops of the composition is administered to the eye of the subject up to three times daily, e.g., once, twice or three times daily. In some embodiments, one or more drops of the composition is administered to the eye of the subject up to three times daily, up to three times every other day, up to three times every three days, up to three times every four days, up to three times every five days, up to three times every week or longer. In some embodiments, one or more drops of the composition is administered to the eye of the subject up to three times daily for at least 5 days, at least 1 week, at least 2 weeks, at least 3 weeks, at least 4 weeks, at least 5 weeks, at least 6 weeks, at least 7 weeks, at least 8 weeks, at least 10 weeks, at least 3 months, at least 4 months, at least 6 months, at least 8 months, at least 12 months, at least 18 months, at least 24 months or longer, or for a length of time in a range defined by any two of the preceding values (e.g., 5 days to 24 months, 1 week to 24 months, 1-6 weeks, 1 week to 12 months, 6-10 weeks, 3-12 months, etc.).

In some embodiments, the composition is administered to one eye of the subject, e.g., to the eye that exhibits one or more of the moderate/severe dry eye-related ocular symptoms. In some embodiments, the composition is administered to both eyes of the subject. In some embodiments, the composition is administered to one eye of the subject, e.g., to the eye that exhibits one or more of the severe dry eye-related ocular symptoms. In some embodiments, the composition is administered to both eyes of the subject. In some embodiments the subject does not have Sjögren's Syndrome (Primary or Secondary).

In some embodiments, the subject is identified as suffering from severe dry eye, rather than moderate/severe dry eye. In some embodiments, the subject has severe dry eye and/or dry eye symptoms when the subject has Eye Dryness score of >70 using the VAS instantaneous measure in at least one eye.

In some embodiments, the administration of the composition to the eye improves one or more patient reported symptoms or clinical signs of moderate/severe dry eye. In some embodiments the subject does not have Sjögren's Syndrome (Primary or Secondary). Improvements in dry eye symptoms or signs can be assessed by one or more of the following.
Fluorescein corneal staining (FCS) (0 to 3 scale by region, for 5 regions, total 0-15 scale, using the NEI/Industry Workshop scale)
Lissamine green conjunctival staining (LGCS) (0 to 3 scale by region, total 0-18 scale, using NET/Industry Workshop scale)
Anesthetized Schirmer test (mm of wetting in 5 minutes),
Tear film break-up time (number of seconds)
Eye dryness as reported by the patient on a visual analog scale and tabulated as a mean change from baseline
Dry eye-related ocular symptoms questionnaire (SANDE: how frequent and how severe are dry eye symptoms), (Schaumberg D, et al. Development and Validation of a Short Global Dry Eye Symptom Index. The Ocular Surface. January 2007, Vol 5; 1; 50-57, incorporated herein by reference in its entirety).

In some embodiments, assessing improvement in moderate/severe or severe dry eye symptoms or signs includes reflective and/or instantaneous assessment of symptoms. In some embodiments, reflective assessment includes rating the difference in symptoms experienced by the subject after treatment compared to before treatment. In some embodiments, reflective assessment of symptoms includes rating the difference in symptoms experienced by the subject in the present compared to the last day of treatment (e.g., the first day of treatment). In some embodiments, reflective assessment of symptoms includes rating the difference in symptoms experienced by the subject in the present compared to the last day of assessment of dry eye symptoms. In some embodiments, the improvement in the measure or assessment of the symptom or sign of dry eye (e.g., those listed above) is, is about, is at least, is at least about, 5, 10, 15, 20, 25, 30, 35, 40, 45, 50, 55, 60, 65, 70, 75, 80, 85, 90, 95, or 100% of the value of the measure or assessment, or a range defined by any two of the preceding values.

In some embodiments, the composition administered to the subject suffering from moderate/severe dry eye symptoms is a sterile aqueous composition comprising, consisting or consisting essentially of a composition disclosed herein. In some embodiments, the composition is a sterile aqueous composition comprising, consisting or consisting essentially of a formulation in Table A, B or C. In some embodiments, the composition is a sterile aqueous composition comprising, consisting or consisting essentially of Formula A in Table A. In some embodiments, the composition is a sterile aqueous composition comprising, consisting or consisting essentially of Formula B in Table A. In some embodiments, the composition is a sterile aqueous composition comprising, consisting or consisting essentially of Formula C in Table A. In some embodiments, the composition is a sterile aqueous composition comprising, consisting or consisting essentially of Formula D in Table B. In some embodiments, the composition is a sterile aqueous composition comprising, consisting or consisting essentially of Formula E in Table B. In some embodiments, the composition is a sterile aqueous composition comprising, consisting or consisting essentially of Formula F in Table B. In some embodiments, the composition is a sterile aqueous composition comprising, consisting or consisting essentially of Formula G in Table C. In some embodiments, the composition is a sterile aqueous composition comprising, consisting or consisting essentially of Formula H in Table C. In some embodiments, the composition is a sterile aqueous composition comprising, consisting or consisting essentially of Formula 1 in Table C.

Some embodiments include a method of treating moderate/severe dry eye comprising administering a composition disclosed herein to the eye of a subject having moderate/severe dry eye. In some embodiments, the subject does not have Sjögren's Syndrome (Primary or Secondary). In some embodiments, the compositions described herein are used to treat a subject with moderate/severe dry eye symptoms. In some embodiments the subject is identified as moderate/severe dry eye by meeting 1, 2, 3 or 4 of the following criteria:
a) FCS total score ≥4 and <15 in the NEI/Industry Workshop scale,
b) Eye Dryness score of ≥60 using the VAS instantaneous,
c) Anesthetized Schirmer 1 test score ≤5 mm wetting/5 min, and
d) LGCS total score ≥5 using the NEI/Industry Workshop scale (where 0=no staining).

In some embodiments, the compositions described herein are used to treat subjects that have all four of the criteria from the list above. In some embodiments, the compositions described herein are used to treat subjects that have all four of the criteria from the list above in at least one eye (e.g., in one or both eyes). In some embodiments, the subject does not have Sjögren's Syndrome (Primary or Secondary). In some embodiments, the subject does not meet 1, 2, 3, or 4 of the following criteria in one eye, or in both eyes: a) FCS total score <4 or ≥15 in the NEI/Industry Workshop scale; b) Eye Dryness score of <60 using the VAS instantaneous; c) Anesthetized Schirmer 1 test score >5 mm wetting/5 min; and d) LGCS total score <5 using the NEI/Industry Workshop scale (where 0=no staining). In some embodiments, the polypeptide or pharmaceutically acceptable salt thereof in the composition is Lacripep™ (having a sequence consisting of Ac-Lys-Gln-Phe-Ile-Glu-Asn-Gly-Ser-Glu-Phe-Ala-Gln-Lys-Leu-Leu-Lys-Lys-Phe-Ser-NH₂, where “Ac” represents an acetyl group and the C-terminus is amidated (SEQ ID NO: 1)) in an amount of 0.005%, 0.001%, or 0.00025% (w/v). In some embodiments, the ophthalmic solution further comprises Citric Acid (about 0.0098% anhydrous), Sodium Citrate (about 0.279% sodium citrate dehydrate), EDTA Disodium (about 0.001%), NaCl (about 0.5%), Tyloxapol (about 0.001%, 0.01%, or 0.05%), NaOH or HCl (to about 6.5 pH), Purified Water, USP in addition to the polypeptide, e.g., Lacripep™. In some embodiments, the subject having moderate/severe dry eye symptoms is administered a sterile aqueous composition comprising, consisting or consisting essentially of Formula A, B, C, D, E, F, G, H, or I in Tables A, B, and C, or Formulas 1, 2, 3, 4, 5, 6, 7, 8, or 9 of Tables 1.1, 1.2, and 1.3. For assessing efficacy, some embodiments utilize a placebo comprising a vehicle ophthalmic solution without the polypeptide. In some embodiments one drop of the composition is administered to the eye of the subject up to three times daily. In some embodiments, the subject is identified as suffering from severe dry eye, rather than moderate/severe dry eye. In some embodiments, the subject has severe dry eye and/or dry eye symptoms when the subject has Eye Dryness score of >70 using the VAS instantaneous measure in at least one eye.

In some embodiments, the method includes measuring a change in the one or more dry eye-related ocular symptoms after administering compared to before. In some embodiments, the method includes measuring scores for one or more of eye dryness (EDS), SANDE, LGCS, FCS, the Schirmer test (e.g., anesthetized Schirmer test), and TBUT, after administering, and comparing with the corresponding score before administering.

In some embodiments, the administration improves the FCS total score (NET/Industry Workshop 0-15 scale) in the subject's eye after at least two weeks of treatment, or after at least four weeks of treatment, or after at least six weeks from the start of four weeks of treatment, compared to a baseline measure prior to starting treatment. In some embodiments, the administration improves one or more of:
eye dryness after at least two weeks of treatment, or after at least four weeks of treatment, compared to baseline on a visual analog scale;
SANDE (global scores SANDE 1) after at least two weeks of treatment compared to a baseline measure prior to starting treatment;
Mean Scores for SANDE (global scores SANDE-1) after at least two weeks of treatment compared to a baseline measure prior to starting treatment;
Individual Symptom Assessments (Instantaneous) after at least two weeks of treatment compared to a baseline measure prior to starting treatment;
Mean Scores for Individual Symptom Assessments (Reflective) after at least two weeks of treatment compared to a baseline measure prior to starting treatment;
LGCS in the subject's eye after at least two weeks of treatment compared to a baseline measure prior to starting treatment;
Anesthetized Schirmer test in the subject's eye after at least two weeks of treatment compared to a baseline measure prior to starting treatment;
TFBUT in the subject's eye after at least two weeks of treatment compared to a baseline measure prior to starting treatment;
FCS in the subject's eye after at least two weeks of treatment compared to a baseline measure prior to starting treatment;
SANDE (global scores for SANDE 1) after at least 2 weeks of treatment, or after at least 4 weeks of treatment, or 1 week after 4 weeks treatment compared to a baseline measure prior to starting treatment;
Individual Symptoms (Instantaneous) after at least 2 weeks of treatment, or after at least 4 weeks of treatment, or 1 week after 4 weeks treatment compared to a baseline measure prior to starting treatment;
Mean Scores for (global scores SANDE-2) after at least 2 weeks of treatment, or after at least 4 weeks of treatment, or 1 week after 4 weeks treatment compared to a baseline measure prior to starting treatment;
Mean Scores for Individual Symptom Assessments (Reflective) after at least 2 weeks of treatment, or after at least 4 weeks of treatment, or 1 week after 4 weeks treatment compared to a baseline measure prior to starting treatment,
FCS and SANDE 1 and Individual Symptom Assessments (Instantaneous) after at least 2 weeks of treatment, or after at least 4 weeks of treatment, compared to a baseline measure prior to starting treatment;
LGCS after at least 2 weeks of treatment, or after at least 4 weeks of treatment compared to a baseline measure prior to starting treatment,
Anesthetized Schirmer test results after at least 2 weeks of treatment, or after at least 4 weeks of treatment, compared to a baseline measure prior to starting treatment;
TFBUT after at least 2 weeks of treatment, or after at least 4 weeks of treatment, or 1 week after 4 weeks treatment compared to a baseline measure prior to starting treatment.

In some embodiments, the improvement in the measure or assessment of the symptom or sign of dry eye (e.g., those listed above) is, is about, is at least, is at least about, 5, 10, 15, 20, 25, 30, 35, 40, 45, 50, 55, 60, 65, 70, 75, 80, 85, 90, 95, or 100% of the value of the measure or assessment, or a range defined by any two of the preceding values.

In some embodiments, the comparison instead, or further, comprises a comparison to a vehicle control.

In some embodiments, any one or more of the following drugs/therapies are not co-administered with the compositions described herein, and in some embodiments any one or more of the following drugs/therapies are co-administered with the with the compositions described herein:
1. Ophthalmic drugs (any topical eye medications) including prescription medication and over-the-counter [OTC] agents
2. Contact lenses
3. Any ocular surface or eyelid operative procedure within 365 days prior to start of treatment or intraocular surgery within 90 days prior to start of treatment.
4. amiodarone.
5. topical ocular antihistamines
6. ocular, inhaled or intranasal corticosteroids
7. topical or oral mast cell stabilizers
8. oral antihistamines
9. topical or nasal vasoconstrictors
10. topical ocular NSAIDs
11. topical ocular antibiotics
12. Within 60 days prior to and/or during treatment: topical cyclosporine, topical lifitegrast
13. Within 90 days prior to and/or during treatment: cauterization of the punctum or alternations to (insertion or removal) punctal plug(s) or nasolacrimal surgery.
14. Chronic oral anti-viral medications for ocular herpetic disease.

### EXAMPLES

The following are non-limiting examples of some of the embodiments described herein.

### Example 1—Treatment of Moderate/Severe Dry Eye

A randomized, placebo-controlled, double-masked, parallel-group study is conducted to determine the therapeutic effect of 0.005% (22 μM), 0.001% (4 μM), or 0.00025% (1 μM) Lacripep™ in an identified cohort of patients having moderate/severe dry eye-related symptoms.

### Exclusion and Inclusion Criteria

Subjects who meet the following criteria are selected:
1. Subjects who are age 18 years of age or older at the time of obtaining informed consent.
2. Subjects with a history of dry eye-related ocular symptoms, and who have self-reported use of over the counter ocular wetting agents within the last 120 days.
3. Subjects who meet the following criteria at both screening and Visit 2 (Randomization/Baseline) examinations:
a) FCS total score ≥4 and <15 in the NEI/Industry Workshop scale,
b) Eye Dryness score (EDS) of ≥60 using the VAS instantaneous,
c) Anesthetized Schirmer 1 test score <5 mm wetting/5 min,
d) LGCS total score >5 using the NEI/Industry Workshop scale (where 0=no staining).

Note: Subjects must meet all criteria and eligible scores for FCS, EDS, Schirmer and LGCS must be in at least one eye and it must be in the same eye at the time of the visit.

The FCS is scored by a trained ophthalmologist who scores the level of corneal fluorescein staining within a grid that divides the corneal area into five sections (FIG. 7), each of which is assigned a score (or grade) from zero and 3 depending on the amount and distribution of staining. Score/grade 0 indicates no staining in a section. The total FCS score is determined in a range from 0/15 (indicating absence of corneal epitheliopathy) to 15/15 (indicating severe epitheliopathy).

The LGCS is scored by a trained ophthalmologist who scores the level of Lissamine green conjunctival staining within a grid that divides the conjunctiva area into six sections (superior paralimbal, inferior paralimbal, peripheral area both nasally & temporally) (FIG. 8) each of which is assigned a score (or grade) from zero and 3 depending on the amount and distribution of staining. Score/grade 0 indicates no staining in a section. The total FCS score ranges from 0/15 (indicating absence of corneal epitheliopathy) to 15/15 (indicating severe epitheliopathy), each of which is assigned a score (or grade) from zero and 3 depending on the amount and distribution of staining. Score/grade 0 indicates no staining in a section. The total LGCS score is determined in a range from 0/18 to 18/18.

The EDS is scored by instantaneous assessment of symptoms by the subject as reported by the questionnaire shown in FIG. 6. In the questionnaire, the subject is asked to rate their dry eye symptoms by marking an “X” on a line for each. EDS is the value for the “Eye Dryness” line of the questionnaire.

The Schirmer test is performed by folding the Schirmer paper strip (5×35 mm) at the notch and hooking the folded end over the temporal one-third of the lower lid margin. The score is the measured length of wetting from the notch, after a period of 5 min. For the anesthetized Schirmer test score, anesthesia is applied to the subject's eye before applying the Schirmer paper strip.

Subjects meeting any of the following criteria at the Visit 1 (Screening) or Visit 2 (Randomization/Baseline) visits are excluded:
1. Subjects with any active infectious ocular condition.
2. Subjects who are monocular or have a BCVA, using corrective lenses if necessary, of +1.0 logMAR or worse as assessed by Early Treatment Diabetic Retinopathy Study (ETDRS).
3. Subjects with ocular inflammatory conditions (e.g., conjunctivitis, keratitis, anterior blepharitis, etc.) not related to dry eye syndrome.
4. Subjects with clinical evidence of cicatricial ocular surface disease, such as cicatricial ocular pemphigoid or Stevens Johnson syndrome.
5. Subjects who cannot suspend the use of any topical eye medications (including topical cyclosporine) other than the investigational product during the run-in and the study treatment phase.
6. Subjects who have used Restasis® (topical ophthalmic cyclosporine) within 60 days prior to visit 1.
7. Subjects who have used Xiidra® (topical ophthalmic lifitegrast) within 60 days prior to Visit 1.
8. Subjects who in the study eye have fluorescein corneal staining (FCS) Total Score=15 or a Score=3 in the superior region per the NEI/Industry Workshop scale or subjects who have FCS with diffuse confluent staining, filaments or epithelial defects.
9. Subjects who have active or have had an outbreak of herpetic keratitis within 365 days of Visit 1 or subjects who are on chronic oral antivirals for herpetic disease.
10. Subjects who cannot suspend the use of and abstain from contact lens use from the Screening Visit (Visit 1) to the end of the study (Visit 5).
11. Subjects who have used or anticipate use of amiodarone.
12. Subjects who within 30 days prior to Visit 1 alter the dose or anticipate alterations to the dose of the following: tetracyclines, Omega 3's or 6's.
13. Subjects who within 60 days prior to Visit 1 and for the duration of the study alter the dose or anticipate alterations to the dose of the following: anticholinergics, antidepressants, oral contraceptives, isotretinoin, oral systemic corticosteroids, oral systemic immunosuppressive agents.
14. Subjects who within 30 days prior to Visit 1 and for the duration of the study use topical ocular antihistamines, ocular, inhaled or intranasal corticosteroids, topical or oral mast cell stabilizers, oral antihistamines, topical or nasal vasoconstrictors, topical ocular NSAIDs, topical ocular antibiotics.
15. Subjects who in the study eye have had cauterization of the punctum or alterations to (insertion or removal) punctal plug(s) within the past 90 days prior to Visit 1. Note: If a punctal plug in place at Visit 2 (Randomization/Baseline) and it is dislodged, the plug should be replaced as soon as possible.
16. Subjects who, in the study eye, have had corneal refractive surgery (LASIK, PRK, RK).
17. Subjects who in the study eye, have a history of any operative procedure on the ocular surface or eyelids within 365 days prior to Visit 1 or with a history of intraocular surgery within 90 days prior to Visit 1.
18. Subjects who are pregnant or suspected to be pregnant and subjects who are breastfeeding or intend to breastfeed. Female subjects of childbearing potential are required to have a negative urine pregnancy test at screening, and must agree to use an acceptable method of contraception from the time of signing informed consent until the end of study visit. Medically acceptable contraception methods include intrauterine device; barrier methods such as diaphragm, condom, cap or sponge, used with a spermicide; or hormonal contraception.
19. Subjects with any physical or mental impairment that would preclude participation and the ability to give informed consent.
20. Subjects who have participated in a device or Investigational drug study or clinical trial within 30 days of Visit 1. Participation in another during this study is excluded for the duration of this study.

Eligible subjects are randomly assigned to one of four treatment groups: one of three Lacripep™ ophthalmic solution strengths (0.005% (Formula 1 or 4), 0.001% (Formula 2 or 5), or 0.00025% (Formula 3 or 6) w/v) or placebo (respective vehicle). Each study arm includes 65 subjects, for a total of 260 subjects. The Lacripep™ formulations are shown in Table 1.1, Table 1.2, and Table 1.3.

One drop of investigational product is administered three times a day (TID) to both eyes for one, two, four or six weeks. Efficacy and safety are evaluated at predetermined intervals.

TABLE 1.1
Lacripep ™ Formulations

| | | Amount (% w/v) | | |
|---|---|---|---|---|
| Component | Grade | Formula 1 | Formula 2 | Formula 3 |
| Lacripep ™ | | 0.005 (22 μM) | 0.001 (4 μM) | 0.00025 (1 μM) |
| Citric Acid, anhydrous | USP | 0.0098 | 0.0098 | 0.0098 |
| Sodium Citrate, anhydrous | USP | 0.279 | 0.279 | 0.279 |
| Disodium Edetate (EDTA), | USP | 0.001 | 0.001 | 0.001 |
| dihydrate | | | | |
| NaCl | USP | 0.5 | 0.5 | 0.5 |
| Tyloxapol | USP | 0.001 | 0.001 | 0.001 |
| NaOH | USP | as needed* | as needed* | as needed* |
| HCl | USP | as needed* | as needed* | as needed* |
| Water for Injection | USP | QS | QS | QS |

*pH 6.3 to 6.5 (Target = 6.5)

TABLE 1.2
Lacripep ™ Formulations

| | | Amount (% w/v) | | |
|---|---|---|---|---|
| Component | Grade | Formula 4 | Formula 5 | Formula 6 |
| Lacripep ™ | | 0.005 (22 μM) | 0.001 (4 μM) | 0.00025 (1 μM) |
| Citric Acid, anhydrous | USP | 0.0098 | 0.0098 | 0.0098 |
| Sodium Citrate, anhydrous | USP | 0.279 | 0.279 | 0.279 |
| Disodium Edetate (EDTA), | USP | 0.001 | 0.001 | 0.001 |
| dihydrate | | | | |
| NaCl | USP | 0.5 | 0.5 | 0.5 |
| Tyloxapol | USP | 0.01 | 0.01 | 0.01 |
| NaOH | USP | as needed* | as needed* | as needed* |
| HCl | USP | as needed* | as needed* | as needed* |
| Water for Injection | USP | QS | QS | QS |

*pH 6.3 to 6.5 (Target = 6.5)

TABLE 1.3
Lacripep ™ Formulations

| | | Amount (% w/v) | | |
|---|---|---|---|---|
| Component | Grade | Formula 7 | Formula 8 | Formula 9 |
| Lacripep ™ | | 0.005 (22 μM) | 0.001 (4 μM) | 0.00025 (1 μM) |
| Citric Acid, anhydrous | USP | 0.0098 | 0.0098 | 0.0098 |
| Sodium Citrate, anhydrous | USP | 0.279 | 0.279 | 0.279 |
| Disodium Edetate (EDTA), | USP | 0.001 | 0.001 | 0.001 |
| dihydrate | | | | |
| NaCl | USP | 0.5 | 0.5 | 0.5 |
| Tyloxapol | USP | 0.05 | 0.05 | 0.05 |
| NaOH | USP | as needed* | as needed* | as needed* |
| HCl | USP | as needed* | as needed* | as needed* |
| Water for Injection | USP | QS | QS | QS |

*pH 6.3 to 6.5 (Target = 6.5)

Efficacy: All efficacy assessments are performed in both eyes, and by the same person if possible. Corneal Fluorescein Staining (CFS) is assessed in 5 regions (Central, Inferior, Superior, Temporal, and Nasal) on a 0 to 3 scale (total 0 to 15). Lissamine green conjunctival staining (LGCS) is assessed in 6 regions on a 0 to 3 scale (total 0 to 18). All sites are trained and tested on NEI scoring methodology.

The anesthetized Schirmer test was performed after instilling ˜50 μL (one drop) of 0.5% proparacaine after drying the inferior cul-de-sac and recorded in mm of wetting over 5 minutes. Tear film break-up time (TBUT) was measured in seconds. The Symptom Assessment in Dry Eye (SANDE) inventory version 1 and version 2 measured patient-rated symptom severity and frequency using a visual-analogue scale. The Individual Symptom Assessments (instantaneous and Reflective) measured patient-reported symptoms on a visual-analogue scale (VAS). The SANDE version 2 and Individual Symptom Assessments (Reflective) asked patients to rate the difference in their symptoms as compared to their last clinic visit, and were administered to subjects at Day 14, 28 and 42 of treatment.

The primary efficacy measurement is mean change from Baseline to Day 28 in CFS total score (NEI/Industry Workshop 0-15 scale, 0-3 scale in each of 5 regions) in the study eye. The key secondary efficacy measurement is mean change from Baseline to Day 28 in Eye Dryness Score (VAS from 0 to 100 mm) from Individual Symptom Assessments.

The change from Baseline in CFS to Day 14 and to post treatment follow-up (Day 42) are additional secondary measurements. Other secondary measurements include changes from baseline to Day 14 and Day 28 in the LGCS scores, TFBUT, and Schirmer tests. Secondary symptom measurements include changes from baseline to Day 14 and Day 28 in the SANDE and each of the Individual Symptom Assessments.

The results of the study will show a significant improvement in one or more symptoms relative to the vehicle treatment.

### Example 2—Treatment of Primary Sjögren's Syndrome

The primary objective of this study was to evaluate the safety and tolerability of 22 and 44 μM Lacripep™ ophthalmic solution versus placebo (vehicle) administered three times daily for 28 days in subjects with a history of ocular surface disease associated with Primary Sjögren's Syndrome, including subjects with mild dry eye symptoms. Additionally, many dry eye signs and symptoms were assessed.

Lacripep™ is a synthetic 19 amino acid peptide fragment of lacritin (SEQ ID NO: 1). Proteomic studies have revealed the active, monomeric form of lacritin to be downregulated in tears of patients suffering from many forms of dry eye disease, including aqueous deficient, evaporative, contact lens-related but most strikingly in dry eye patients with Primary and Secondary Sjögren's Syndrome.

### Study Design

This was a multi-center, randomized, placebo-controlled, double-masked, parallel-group study conducted at 35 sites in the United States, and approved by local institutional review boards. Written informed consent was obtained from all subjects after review of risks and benefits of participation. The study was conducted in accordance with the Health Insurance Portability and Accountability Act of 1996 and Declaration of Helsinki of 1996 and registered at ClinicalTrials.gov: NCT 03226444.

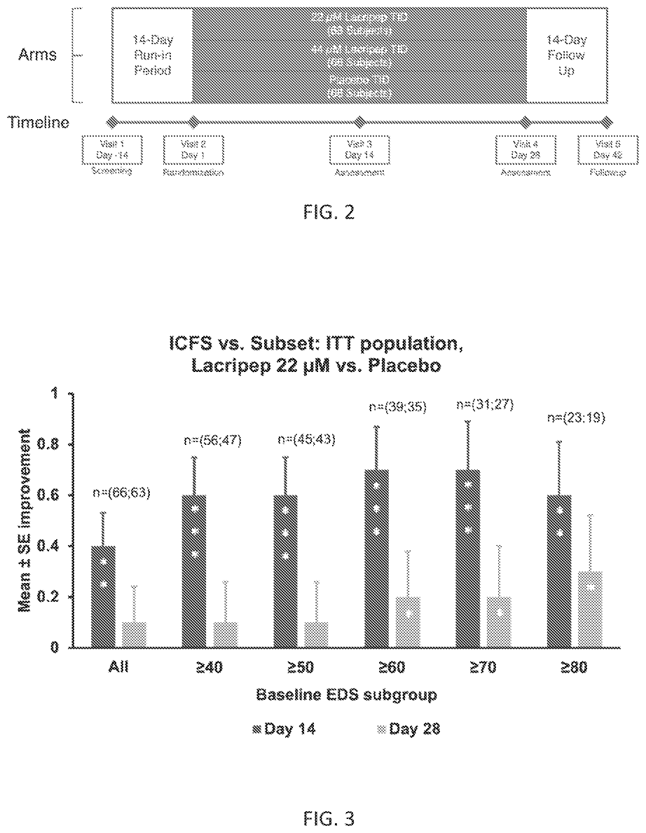

The study duration was 56 days, including a 14-day run-in period, a 28-day active treatment period, and 14-day follow-up period (wash-out) (see FIG. 2). Measurements were collected at five scheduled visits.

### Study Protocol

Screening and Eligibility: At Visit 1 (Screening) informed consent was obtained from subjects and eligibility was determined requiring documentation of Primary Sjögren's Syndrome per the American-European Consensus Group Sjögren's Syndrome Criteria. Subjects were at least 18 years of age with a history of dry eye-related symptoms and use of eye-wetting agents within the past 120 days. Major inclusion criteria at the screening and subsequent baseline visits included:
1. CFS total score >4 and <15 in the National Eye Institute Industry Workshop (NEI) scale (Protocol Appendix 6)
2. Symptom Severity score of ≥40 using the Symptom Assessment in Dry Eye (SANDE) questionnaire
3. Anesthetized Schirmer test score 5 mm wetting/5 min
4. LGCS total score ≥5 using the NEI scale

Subjects with the following were excluded.
1. Active infectious ocular condition.
2. Ocular inflammatory conditions not related to dry eye syndrome.
3. Clinical evidence of cicatricial ocular surface disease
4. Use of Restasis® (topical ophthalmic cyclosporine) or Xiidra® (topical ophthalmic lifitegrast) within 14 days prior to Visit 1.
5. A history of collagen vascular disease, auto immune disease, or rheumatic disease other than Primary Sjögren's Syndrome (e.g., Lupus, Rheumatoid Arthritis, etc.)
6. A history of or current Anterior Membrane Dystrophy, corneal transplantation, corneal refractive surgery, or other recent ocular procedures.
7. Childbearing potential unwilling to use contraception, or pregnant or breastfeeding.
8. Any physical or mental impairment that would have precluded participation and the ability to give informed consent.

Those eligible entered a 14-day run-in period involving instillation of one drop of single-masked placebo (vehicle) three times a day to each eye.

Randomization and Treatment: At Visit 2 (Baseline/Randomization) eligibility was confirmed. All criteria were met in the same study eye. For each subject, the study eye was the one qualifying for study inclusion, or the one with higher baseline corneal fluorescein staining total score if both qualified, or the right eye if both eyes showed the same baseline score. Eligible subjects were randomly assigned to one of the three treatment groups: one of two Lacripep™ ophthalmic solution strengths (22 μM or 44 μM) or placebo (vehicle).

One drop of investigational product was administered three times a day (TID) to both eyes for 28 days. At Visit 3 (Week 2) and Visit 4 (Week 4) efficacy and safety evaluations were performed. Subjects who discontinued before Visit 4 underwent Visit 4 evaluations (Early Termination).

14-Day Follow-Up Period: After discontinuation of investigational product, there was a 14-day follow-up period during which subjects instilled one drop of Refresh Plus® (Allergan, Dublin Ireland) TID to each eye.

At Visit 5 (Week 6 follow-up) efficacy and safety evaluations were performed.

Concomitant medications/therapies: Subjects whose records indicated the use of prohibited medications (topical, topical ophthalmic, systemic and/or injectable) during the appropriate pre-study washout period and/or during the 14-day vehicle run-in period prior to randomization were excluded from efficacy analyses prior to database lock.

Subjects had not received any investigational drug or device within 30 days of screening, nor during the study except per-protocol. Subjects who were on systemic (oral) therapy for the treatment of Sjögren's Syndrome must have been on stable systemic treatment defined as the same treatment for the immediately prior 90 days. The use of cyclosporin (compounded or Restasis® Allergan, Irvine CA or Cequa, Sun Pharmaceuticals, Mumbai, India) or lifitegrast (Xiidra®, Novartis, Basel, CH) within 14 days prior to the screening examination was prohibited. Subjects did not have alterations to (insertion or removal) punctal plugs in the study eye, within 14 days prior to the screening examination and during the entire study.

Medications topical or systemic, known to exacerbate dry eye were prohibited during the study.

Study Masking: Subjects were randomly assigned to receive either placebo or study drug. The placebo (vehicle) and Lacripep™ containers during the double-masked treatment phase were identical in appearance. Study subjects and investigators and their staff were masked to the identity of treatment until the final database was locked.

### Outcome Measures

Efficacy: All efficacy assessments were performed in both eyes, and by the same person if possible. Corneal Fluorescein Staining (CFS) was assessed in 5 regions (Central, Inferior, Superior, Temporal, and Nasal) on a 0 to 3 scale (total 0 to 15). Lissamine green conjunctival staining (LGCS) was assessed in 6 regions on a 0 to 3 scale (total 0 to 18). All sites were trained and tested on NEI scoring methodology.

The anesthetized Schirmer test was performed after instilling ˜50 μL (one drop) of 0.5% proparacaine after drying the inferior cul-de-sac and recorded in mm of wetting over 5 minutes. Tear film break-up time (TBUT) was measured in seconds. The Symptom Assessment in Dry Eye (SANDE) inventory version 1 and version 2 measured patient-rated symptom severity and frequency using a visual-analogue scale. The Individual Symptom Assessments (instantaneous and Reflective) measured patient-reported symptoms on a visual-analogue scale (VAS). The SANDE version 2 and Individual Symptom Assessments (Reflective) asked patients to rate the difference in their symptoms as compared to their last clinic visit, and were administered to subjects at Day 14, 28 and 42 of treatment.

The primary efficacy measurement was mean change from Baseline to Day 28 in CFS total score (NEI/Industry Workshop 0-15 scale, 0-3 scale in each of 5 regions) in the study eye. The key secondary efficacy measurement was mean change from Baseline to Day 28 in Eye Dryness Score (VAS from 0 to 100 mm) from Individual Symptom Assessments.

The change from Baseline in CFS to Day 14 and to post treatment follow-up (Day 42) were additional secondary measurements. Other secondary measurements included changes from baseline to Day 14 and Day 28 in the LGCS scores, TFBUT, and Schirmer tests. Secondary symptom measurements included changes from baseline to Day 14 and Day 28 in the SANDE and each of the Individual Symptom Assessments.

Safety: Safety measurements included external eye exam, dilated ophthalmoscopy exam, intraocular pressure (IOP), slit lamp biomicroscopy, and best-corrected visual acuity (BCVA). If a subject had a diagnosis of or was noted to have Meibomian Gland Disease (MGD), the severity was rated.

All treatment-emergent adverse events (TEAEs), their severity, and relatedness to study drug were recorded.

Statistical Analysis: All analysis was performed using SAS (version 9.4, SAS Institute, Cary NC).

Analysis Sets: The intent-to-treat (TTT) set included all subjects who took at least one dose of investigational product, as indicated on the dosing record. All safety variables were analyzed using the safety analysis set and only observed data were included (i.e., missing data remained missing for the safety analysis).

The full analysis (FAS) set consisted of all subjects in the ITT set who met the pre-specified inclusion criteria. Prior to database lock, several subjects were excluded from the FAS based on prospective eligibility exclusions and/or prospectively defined protocol violations.

Baseline and Safety Analyses: Demographic and baseline efficacy assessment parameters were summarized by treatment group. Baseline ocular assessments were summarized for the study eyes. Adverse events were classified according to the Medical Dictionary for Regulatory Activities (MedDRA version 20.0. MedDRA MSSA, McLean, VA). Adverse events, and any adverse findings of standard safety examinations, were enumerated. Adverse event counts and percentages were summarized by treatment group, as well as the existence of any serious adverse events. Safety data was examined for trends among the treatment groups.

Prespecified Efficacy Analysis: The prespecified primary and secondary endpoint analysis was performed on the FAS.

The primary endpoints, difference between placebo and each of the two active dose groups in change from baseline to Day 28 in CFS total score, were tested first. A Bonferroni correction was employed to control overall Type 1 error (i.e., these were formally tested to a significance level of p<0.025). The key secondary endpoints (Eye Dryness Score) were similarly tested using a Bonferroni gatekeeping procedure, i.e., if the primary endpoints were met.

Next, the key secondary analysis was performed, with statistical inference contingent upon the primary inference via the Bonferroni gatekeeper procedure. Other secondary efficacy endpoints were examined for any trends among treatment groups. All inferential summaries for these analyses were used for descriptive purposes. The point estimate and 95% confidence interval (CI) for the treatment differences were calculated along with the p-value for the treatment comparison using a t-test.

All two-sample t-tests conducted in the analyses did not assume equal variances and the Satterthwaite approximation was used for different sample sizes compared. The last observation carried forward (LOCF) was used to impute missing values prior to Day 28, except in the reflective individual symptom assessment, in which the patient explicitly compares symptoms to prior visits.

Post-Hoc Efficacy Analysis: Post-hoc, all primary and secondary endpoints were assessed for treatment effect controlling for baseline Eye Dryness Score (EDS) using analysis of covariance (ANCOVA). All ANCOVA models were fit using the Proc Mixed procedure in SAS. Post-hoc analysis was also performed using all observed data in the ITT set, without imputation. Data from the ITT set was examined for trends and for notable efficacy signals, using endpoint means t standard errors, and p-values from nonparametric Wilcoxon ranked-sum tests as a descriptor of statistical strength. Subgroups were then evaluated according to baseline EDS.

### Results

Subject Disposition: The study screened 350 subjects of which 204 subjects were enrolled for treatment, 68 in each of the three treatment groups, (the ITT set). The FAS excluded 27 subjects based on exclusion criteria or protocol violations. Excluded treatment subjects reported use of prohibited medications such as cyclosporin or lifitegrast during the run-in or displayed signs of comorbid autoimmune/connective tissue diseases characteristic of Secondary Sjögren's.

The FAS consisted of 177 subjects with 60 in the placebo, 57 in the 22 μM Lacripep™ and 60 in the Lacripep™ 44 μM groups. Five subjects in the efficacy analysis set failed to complete the study: three failed to complete the 28-day course of active treatment, and two failed to complete the follow-up period. In prespecified analyses, missing values from the patients withdrawing early were imputed using LOCF.

Subject Demographics: A detailed summary of demographic data is found in Table 2.1. Females comprised 96% of the subjects. Subject mean age was 60 years with 87% identifying as white, the majority not Hispanic or Latino. There were no significant differences in demographics between the three study groups.

Baseline disease characteristics are summarized in Table 2.1. The subjects reported a baseline CFS total score of 9.0±2.7 (mean±SD). The baseline Eye Dryness Score was 65.5±25.5 (mean±SD). All three groups had similar baseline disease characteristics. Similar baseline characteristics were observed for the ITT set and FAS.

TABLE 2.1
Demographic Information and Baseline Characteristics
by Treatment Groups - Intent-to-treat Set

| | | |
|---|---|---|
| | Lacripep ™ 22 | Lacripep ™ 44 |
| Placebo | μM | μM |
| (n = 68) | (n = 68) | (n = 68) |

Age (years)

| | | | |
|---|---|---|---|
| Mean ± SD | 60.0 ± 10.9 | 60.8 ± 11.2 | 60.1 ± 10.7 |

Gender

| | | | | | | |
|---|---|---|---|---|---|---|
| Female | 65 | (95.6%) | 65 | (95.6%) | 66 | (97.1%) |
| Male | 3 | (4.4%) | 3 | (4.4%) | 2 | (2.9%) |

Race

| | | | | | | |
|---|---|---|---|---|---|---|
| White | 59 | (86.8%) | 58 | (85.3%) | 60 | (88.2%) |
| Black or | 4 | (5.9%) | 4 | (5.9%) | 2 | (2.9%) |
| African American | | | | | | |
| Asian | 2 | (2.9%) | 4 | (5.9%) | 4 | (5.9%) |
| Native Hawaiian | 1 | (1.5%) | 0 | (0%) | 0 | (0%) |
| or Other Pacific | | | | | | |
| Islander | | | | | | |
| American Indian or | 2 | (2.9%) | 2 | (2.9%) | 2 | (2.9%) |
| Alaskan Native | | | | | | |

Ethnicity

| | | | | | | |
|---|---|---|---|---|---|---|
| Hispanic or Latino | 8 | (11.8%) | 2 | (2.9%) | 5 | (7.4%) |
| Not Hispanic or Latino | 59 | (86.8%) | 66 | (97.1%) | 61 | (89.7%) |
| Unknown | 1 | (1.5%) | 0 | (0%) | 2 | (2.9%) |

Baseline CFS Total Score

| | | | |
|---|---|---|---|
| Mean ± SD | 9.1 ± 2.8 | 9.3 ± 2.6 | 8.6 ± 2.8 |

Baseline Eye Dryness Score* (mm)

| | | | |
|---|---|---|---|
| Mean ± SD | 65.6 ± 24.9 | 64.8 ± 25.6 | 66.0 ± 25.9 |

Baseline SANDE Version 1 Symptom Severity Score (mm)

| | | | |
|---|---|---|---|
| Mean ± SD | 68.2 ± 16.5 | 69.5 ± 16.9 | 68.1 ± 17.2 |

Baseline LGCS Total Score

| | | | |
|---|---|---|---|
| Mean ± SD | 11.6 ± 4.4 | 12.0 ± 4.3 | 11.8 ± 4.0 |

Baseline Anesthetized Schirmer Test Score (mm)

| | | | |
|---|---|---|---|
| Mean ± SD | 2.4 ± 1.8 | 2.3 ± 1.8 | 2.4 ± 1.5 |

Post-Hoc Efficacy Analysis: The secondary endpoints yielded potential signals of treatment effect in the inferior region CFS changes and the Burning/Stinging symptom changes. These were repeated with ANCOVA analysis using the ITT set, showing a highly significant effect of baseline EDS. To illustrate, FIG. 3 shows the inferior region CFS (ICFS) for the whole ITT set and for subgroups with baseline EDS from ≥40 to ≥80. The baseline EDS≥60 subgroup showed the strongest treatment effect, while preserving 74/129 subjects (57%) in the sample set. The endpoints were re-evaluated in the subset of ITT subjects with EDS 60 at baseline. A significant treatment effect was seen in total and inferior CFS, the Burning/Stinging symptom, and in sub-regions of the conjunctiva using LGCS.

FIG. 3: In post-hoc analysis, inferior Corneal Fluorescein Staining (ICFS) score was computed for subsets of the ITT population according to baseline Eye Dryness Score (EDS). Mean and standard error of the results are plotted for each subset. Numbers of patients in each subset are indicated above the bars. The EDS≥60 subgroup showed the statistically strongest effects (p=0.0001 at 14 days and p=0.026 at day 28). For their descriptive value, p-value levels from post-hoc Wilcoxon tests are indicated within bars (*p<0.05, **p<0.005, ***p<0.0005).

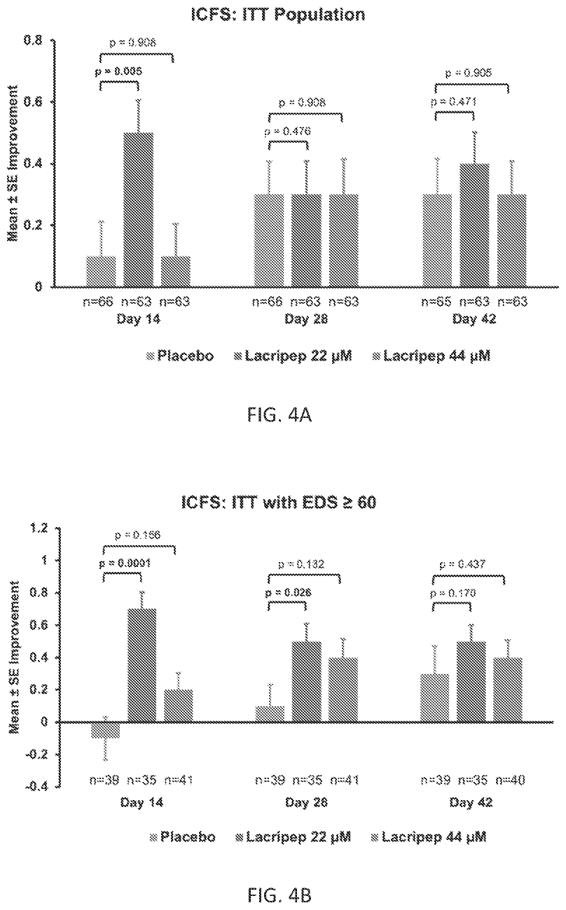

Corneal Staining: Improvement from baseline to day 14 in inferior region CFS score was statistically significant in the prespecified analysis in the 22 μM Lacripep™ group vs. placebo. The post-hoc analysis shows a 0.4 improvement, p=0.005 (see FIG. 4A). In the subset of patients with baseline EDS≥60, the effect is more pronounced (0.8 improvement, p=0.0001) and a significant effect is seen at day 28 as well (0.4 improvement, p=0.026). Positive (though not statistically significant) differences are seen in the higher dose group (see FIG. 4B).

In the baseline EDS 260 set, the prespecified primary endpoint of total CFS score is significantly improved (1.2, p=0.03) at day 14 for the 22 μM dose group vs placebo.

FIGS. 4A, 4B: Post-hoc analysis of the CFS sign. FIG. 4A. The primary endpoint of CFS score at day 28 was not met, but there was a significant treatment effect for the 22 μM dose at 14 days in the inferior corneal region. FIG. 4B. In the subset of subjects with a baseline EDS of 260, significant treatment effect was seen for the 22 μM dose at day 14 and day 28, falling off after washout at day 42; a positive but not significant effect was seen in the 44 μM dose group.

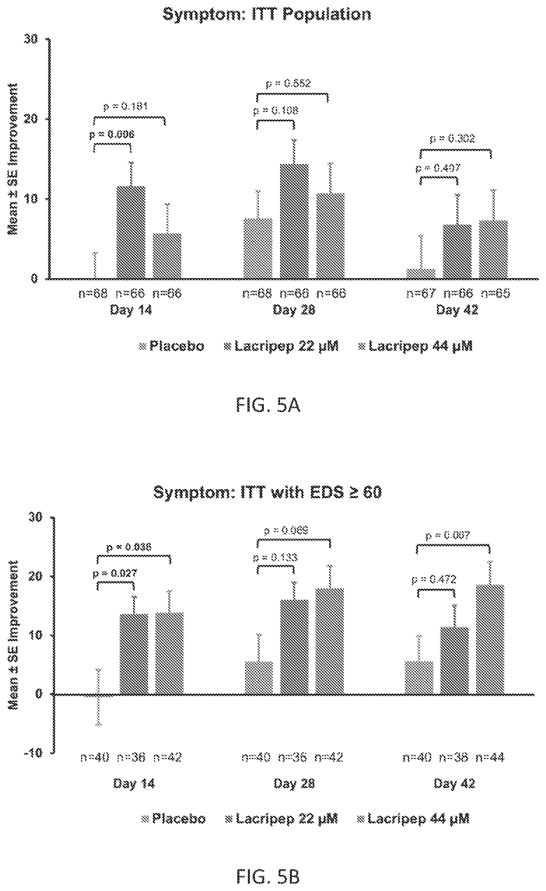

Individual Symptom Assessments (Burning-Stinging): Improvement from baseline to day 14 in the Burning/Stinging symptom (reflective VAS rating) was statistically significant in the prespecified analysis in the 22 μM Lacripep™ group vs. placebo. The post-hoc analysis of the change in instantaneous rating of Burning/Stinging shows a 11.6 mm improvement on the VAS vs. placebo, p=0.006 (see FIG. 5A). In the subset of patients with baseline EDS≥60, a significant effect (14.0 mm improvement, p=0.027) was shown at the same time point, and a significant effect is also shown in the higher dose group (14.2 mm, p=0.038, see FIG. 5B). Both dose groups trend better than the placebo group at day 28 and day 42 (not statistically significant).

FIGS. 5A, 5B. Post-hoc analysis of the Burning/Stinging symptom. FIG. 5A. There was a significant treatment effect for the 22 μM dose at 14 days in the Burning/Stinging symptom. FIG. 5B. In the subset of subjects with a baseline EDS of ≥60, significant treatment effect was seen for both doses at day 14. A positive but not significant effect was seen in both dose groups at day 28.

Lissamine Staining: Improvement in LGCS were not statistically significant in the prespecified secondary endpoint analysis. However, in the subset of patients with baseline EDS≥60, significant effects were seen. The change from baseline LCGS total score was improved for the 22 μM Lacripep™ group vs. placebo at day 14 by 1.5 (p=0.017). The same dose and timepoint showed a significant improvement in segment 1 (0.4, p=0.049) and segment 5 (0.4, p=0.038).

Furthermore, although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the embodiments of the invention(s).

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like.

The indefinite article “a” or “an” does not exclude a plurality. The use of “about” before a number includes the number itself. For example, “about 5” provides express support for “5”.

What is claimed is:

1. A method of treating a moderate/severe dry eye-related ocular symptom, comprising:
identifying a subject suffering from moderate/severe dry eye-related ocular symptoms; and
administering to the subject a liquid composition comprising:
0.0001-0.005% (w/v) of a polypeptide or a pharmaceutically acceptable salt thereof, the polypeptide having a sequence consisting of Ac-Lys-Gln-Phe-Ile-Glu-Asn-Gly-Ser-Glu-Phe-Ala-Gln-Lys-Leu-Leu-Lys-Lys-Phe-Ser-NH₂ (SEQ ID NO: 1), wherein “Ac” represents an acetyl group and the C-terminus is amidated;
0.01-0.6% (w/v) of a buffer;
0.0005-0.01% (w/v) disodium EDTA;
0.0001-0.01% or 0.0001-0.05% (w/v) tyloxapol;
and 0.1-1.0% (w/v) sodium chloride,
wherein the pH of the composition is about 6.2 to about 6.8,
wherein one drop of the composition is administered to an eye of the subject up to three times daily.

2. The method of claim 1, wherein the subject satisfies at least one of the criteria selected from the group consisting of:
a) FCS total score ≥4 and <15 in the NEI/Industry Workshop scale;
b) Eye Dryness score of ≥60 using the VAS instantaneous measure;
c) Anesthetized Schirmer 1 test score <5 mm wetting/5 min; and
d) LGCS total score ≥5 using the NEI/Industry Workshop scale (where 0=no staining).

3. The method of claim 1 or 2, wherein the subject has an Eye Dryness score of ≥60 using the VAS instantaneous measure.

4. The method of claim 1 or 2, wherein the subject satisfies all 4 criteria in a single eye.

5. The method of any one of claims 1-4 wherein the subject does not have Sjögren's Syndrome.

6. The method of any one of the preceding claims, wherein the polypeptide or pharmaceutically acceptable salt thereof is present in the liquid composition at 0.00025-0.005% (w/v), or at 0.0001-0.001% (w/v).

7. The method of any one of the preceding claims, wherein the polypeptide or pharmaceutically acceptable salt thereof is present in the liquid composition at 0.00025% or 0.001% (w/v).

8. The method of any one of the preceding claims, wherein the tyloxapol is present in the liquid composition at 0.0005-0.01% (w/v) or 0.0005-0.05% (w/v).

9. The method of any one of the preceding claims, wherein the tyloxapol is present in the liquid composition at about 0.001% (w/v) or about 0.01% (w/v) or about 0.05% (w/v).

10. The method of any one of the preceding claims, wherein the pH of the composition is about 6.0 to about 7.0, or about 6.4 to about 6.6.

11. The method of claim 10, wherein the pH of the composition is about 6.5.

12. The method of any one of the preceding claims, wherein the amount of NaCl is about 0.4% to about 0.6% (w/v).

13. The method of any one of the preceding claims, wherein the amount of NaCl is about 0.5% (w/v).

14. The method of claim 13, wherein the osmolality of the composition is about 190 to 210 mOsm/kg.

15. The method of any one of the preceding claims, wherein the buffer is a citrate buffer.

16. The method of claim 15, wherein the citrate buffer comprises 0.0098% anhydrous citric acid and 0.279% sodium citrate dihydrate.

17. The method of any one of the preceding claims, wherein the amount of EDTA is about 0.0005% to about 0.005% (w/v).

18. The method of any one of the preceding claims, wherein the amount of EDTA is about 0.001% (w/v).

19. The method of any of any one of the preceding claims, wherein the composition further comprises 0.04% methylparaben.

20. The method of any of any one of the preceding claims, wherein the composition is sterile.

21. The method of any one of the preceding claims, further comprising determining that the subject has a history of one or more dry eye-related ocular symptoms.

22. The method of any one of the preceding claims, wherein the one or more dry eye-related ocular symptoms comprises the subject's use of ocular wetting agents within the last 6 months.

23. The method of any one of the preceding claims, wherein the composition is administered three times daily.

24. The method of any one of the preceding claims, wherein the composition is administered up to three times daily for at least one week.

25. The method of any one of the preceding claims, wherein the composition is administered up to three times daily for 1-6 weeks.

26. The method of any one of the preceding claims, wherein the administration improves the FCS total score (NEI/Industry Workshop 0-15 scale) in the subject's eye after at least two weeks of treatment, or after at least four weeks of treatment, or after at least six weeks from the start of four weeks of treatment, compared to a baseline measure prior to starting treatment.

27. The method of any one of the preceding claims, wherein the administration improves one or more of:
eye dryness after at least two weeks of treatment, or after at least four weeks of treatment, compared to baseline on a visual analog scale,
SANDE (global scores SANDE 1) after at least two weeks of treatment compared to a baseline measure prior to starting treatment;
Mean Scores for SANDE (global scores SANDE-1) after at least two weeks of treatment compared to a baseline measure prior to starting treatment,
Individual Symptom Assessments (Instantaneous) after at least two weeks of treatment compared to a baseline measure prior to starting treatment,
Mean Scores for Individual Symptom Assessments (Reflective) after at least two weeks of treatment compared to a baseline measure prior to starting treatment;
LGCS in the subject's eye after at least two weeks of treatment compared to a baseline measure prior to starting treatment;
Anesthetized Schirmer test in the subject's eye after at least two weeks of treatment compared to a baseline measure prior to starting treatment;
TFBUT in the subject's eye after at least two weeks of treatment compared to a baseline measure prior to starting treatment;
FCS in the subject's eye after at least two weeks of treatment compared to a baseline measure prior to starting treatment;
SANDE (global scores for SANDE 1) after at least 2 weeks of treatment, or after at least 4 weeks of treatment, or 1 week after 4 weeks treatment compared to a baseline measure prior to starting treatment;
Individual Symptoms (Instantaneous) after at least 2 weeks of treatment, or after at least 4 weeks of treatment, or 1 week after 4 weeks treatment compared to a baseline measure prior to starting treatment;
Mean Scores for (global scores SANDE-2) after at least 2 weeks of treatment, or after at least 4 weeks of treatment, or 1 week after 4 weeks treatment compared to a baseline measure prior to starting treatment;
Mean Scores for Individual Symptom Assessments (Reflective) after at least 2 weeks of treatment, or after at least 4 weeks of treatment, or 1 week after 4 weeks treatment compared to a baseline measure prior to starting treatment;
FCS and SANDE 1 and Individual Symptom Assessments (Instantaneous) after at least 2 weeks of treatment, or after at least 4 weeks of treatment, compared to a baseline measure prior to starting treatment;
LGCS after at least 2 weeks of treatment, or after at least 4 weeks of treatment compared to a baseline measure prior to starting treatment,
Anesthetized Schirmer test results after at least 2 weeks of treatment, or after at least 4 weeks of treatment, compared to a baseline measure prior to starting treatment;
TFBUT after at least 2 weeks of treatment, or after at least 4 weeks of treatment, or 1 week after 4 weeks treatment compared to a baseline measure prior to starting treatment.

28. The method of claim 27, the improvement is, is about, is at least, is at least about, 5, 10, 15, 20, 25, 30, 35, 40, 45, 50, 55, 60, 65, 70, 75, 80, 85, 90, 95, or 100% of the value of the measure or assessment, or a range defined by any two of the preceding values.

29. The method of any one of the preceding claims, wherein the subject further meets all the following criteria:
18 years of age or older;
a documented prior history or current diagnosis of dry eye-related ocular symptoms; and
a history of dry eye-related ocular symptoms, and who has self-reported use of over the counter ocular wetting agents within the last 120 days.

30. The method of any one of the preceding claims, wherein the subject does not meet one or more of the following criteria:
any active infectious ocular condition;
monocular or have a Best Corrected Visual Acuity (BCVA), using corrective lenses if necessary, of +1.0 logMAR or worse as assessed by Early Treatment Diabetic Retinopathy Study (ETDRS);
ocular inflammatory conditions (e.g., conjunctivitis, keratitis, anterior blepharitis, etc.) not related to dry eye syndrome;
clinical evidence of cicatricial ocular surface disease, such as cicatricial ocular pemphigoid or Stevens Johnson syndrome;
cannot suspend the use of any topical eye medications (including topical cyclosporine) other than the investigational product during the run-in and the treatment phase;
has used Restasis® (topical ophthalmic cyclosporine) within 60 days prior to beginning treatment with the composition;
has used Xiidra® (topical ophthalmic lifitegrast) within 60 days prior to beginning treatment with the composition;
the subject's eye has fluorescein corneal staining (FCS) total score=15 or a score=3 in the superior region NEI/Industry Workshop scale or the subject's eye has FCS with diffuse confluent staining, filaments or epithelial defects;
has active or have had an outbreak of herpetic keratitis within 365 days of beginning treatment or subjects who are on chronic oral antivirals for herpetic disease;
cannot suspend the use of and abstain from contact lens use during treatment;
has used or anticipate use of amiodarone;
within 30 days prior to beginning treatment alter the dose or anticipate alterations to the dose of the following: tetracyclines, Omega 3 or Omega 6;
who within 60 days prior to beginning treatment and/or for the duration of treatment, altered the dose or anticipate alterations to the dose of the following: anticholinergics, antidepressants, oral contraceptives, isotretinoin, oral systemic corticosteroids, oral systemic immunosuppressive agents,
within 30 days prior to beginning treatment and/or for the duration of the treatment have used topical ocular antihistamines, ocular, inhaled or intranasal corticosteroids, topical or oral mast cell stabilizers, oral antihistamines, topical or nasal vasoconstrictors, topical ocular NSAIDs, topical ocular antibiotics;
in the subject's eye and within the past 90 days have had cauterization of the punctum or alterations to (insertion or removal) punctal plug(s) before beginning treatment;
in the subject's eye, have had corneal refractive surgery (LASIK, PRK, RK);
has a history of any operative procedure on the ocular surface or eyelids within 365 days prior to beginning treatment with a history of intraocular surgery within 90 days prior to beginning treatment;
is pregnant or suspected to be pregnant;
is breastfeeding or intend to breastfeed;
has any physical or mental impairment that would preclude participation and the ability to give informed consent; and
has participated in a device or investigational drug study or clinical trial within 30 days of beginning treatment.

31. The method of any one of the preceding claims, wherein the subject does not meet at least one of the following criteria in one eye, or in both eyes:
a) FCS total score <4 or ≥15 in the NEI/Industry Workshop scale,
b) Eye Dryness score of <60 using the VAS instantaneous,
c) Anesthetized Schirmer 1 test score ≥5 mm wetting/5 min, or
d) LGCS total score <5 using the NEL/Industry Workshop scale (where 0=no staining).

32. The method of any one of the preceding claims, wherein the subject is identified as suffering from severe dry eye, rather than moderate/severe dry eye.

33. The method of claim 32, wherein the subject has Eye Dryness score of ≥70 using the VAS instantaneous measure in at least one eye.

34. The method of any one of the preceding claims, wherein the composition comprises, consists, or consists essentially of, a composition selected from the compositions of a formula in Tables A, B, C, 1.1, 1.2 and 1.3.

35. A liquid composition for use in treating a moderate/severe dry eye-related ocular symptom in an eye of a subject, the composition comprising:
0.0001-0.006% (w/v) of a polypeptide or a pharmaceutically acceptable salt thereof, the polypeptide having a sequence consisting of Ac-Lys-Gln-Phe-Ile-Glu-Asn-Gly-Ser-Glu-Phe-Ala-Gln-Lys-Leu-Leu-Lys-Lys-Phe-Ser-NH₂ (SEQ ID NO: 1), wherein “Ac” represents an acetyl group and the C-terminus is amidated;
0.01-0.6% (w/v) of a buffer;
0.0005-0.01% (w/v) disodium EDTA;
0.0001-0.01% (w/v) or 0.0001-0.05% (w/v) tyloxapol;
and 0.1-1.0% (w/v) sodium chloride,
wherein the pH of the composition is about 6.2 to about 6.8.

36. The liquid composition of claim 35, wherein the composition comprises, consists, or consists essentially of, a composition selected from the compositions of a formula in Tables A, B, C, 1.1, 1.2 and 1.3.

37. The liquid composition of any one of claims 35-36, wherein the subject satisfies at least one of the criteria selected from the group consisting of;
a) FCS total score ≥4 and <15 in the NEI/Industry Workshop scale;
b) Eye Dryness score of ≥60 using the VAS instantaneous measure;
c) Anesthetized Schirmer 1 test score <5 mm wetting/5 min; and
d) LGCS total score ≥5 using the NEI/Industry Workshop scale (where 0=no staining).

38. The liquid composition of any one of claims 35-37, wherein the subject has an Eye Dryness score of ≥60 using the VAS instantaneous measure.

39. The liquid composition of any one of claims 35-38, wherein the subject satisfies all 4 criteria in a single eye.

40. The liquid composition of any one of claims 35-39, wherein the subject does not have Sjögren's Syndrome.

41. The liquid composition of any one of claims 35-40, wherein the subject does not meet at least one at least one of the following criteria in one eye, or in both eyes:
a) FCS total score <4 or ≥15 in the NEI/Industry Workshop scale,
b) Eye Dryness score of <60 using the VAS instantaneous,
c) Anesthetized Schirmer 1 test score ≥5 mm wetting/5 min, or
d) LGCS total score <5 using the NEI/Industry Workshop scale (where 0=no staining).

42. The liquid composition of any one of claims 35-41, wherein the subject is identified as suffering from severe dry eye, rather than moderate/severe dry eye.

43. The liquid composition of claim 42, wherein the subject has Eye Dryness score of ≥70 using the VAS instantaneous measure in at least one eye.


SEQUENCE LISTING (9 sequences)

SEQ ID NO: 1 - protein, 19 aa, synthetic construct
  source 1..19: note = Optionally with the N-terminus acetylated and the C-terminus amidated
KQFIENGSEFAQKLLKKFS

SEQ ID NO: 2 - protein, 25 aa, synthetic construct
  source 1..25: note = Optionally with the N-terminus acetylated and the C-terminus amidated
KQFIENGSEFAQKLLKKFSLLKPWA

SEQ ID NO: 3 - protein, 138 aa, Homo sapiens
  source 1..138: note = Optionally with the N-terminus acetylated and/or the C-terminus amidated
MKFTTLLFLAAVAGALVYAEDASSDSTGADPAQEAGTSKPNEEISGPAEPASPPETTTTAQETSAAAVQGTAKVTSSRQELNPLKSIVEKSILLTEQALAKAGKGMHGGVPGGKQFIENGSEFAQKLLKKFSLLKPWA

SEQ ID NO: 4 - protein, 119 aa, Homo sapiens
  source 1..119: note = Optionally with the N-terminus acetylated and/or the C-terminus amidated
EDASSDSTGADPAQEAGTSKPNEEISGPAEPASPPETTTTAQETSAAAVQGTAKVTSSRQELNPLKSIVEKSILLTEQALAKAGKGMHGGVPGGKQFIENGSEFAQKLLKKFSLLKPWA

SEQ ID NO: 5 - protein, 114 aa, Homo sapiens
  source 1..114: note = Optionally with the N-terminus acetylated and/or the C-terminus amidated
DSTGADPAQEAGTSKPNEEISGPAEPASPPETTTTAQETSAAAVQGTAKVTSSRQELNPLKSIVEKSILLTEQALAKAGKGMHGGVPGGKQFIENGSEFAQKLLKKFSLLKPWA

SEQ ID NO: 6 - protein, 114 aa, Homo sapiens
  source 1..114: note = Optionally with the N-terminus acetylated and/or the C-terminus amidated
EDASSDSTGADPAQEAGTSKPNEEISGPAEPASPPETTTTAQETSAAAVQGTAKVTSSRQELNPLKSIVEKSILLTEQALAKAGKGMHGGVPGGKQFIENGSEFAQKLLKKFSL

SEQ ID NO: 7 - protein, 109 aa, Homo sapiens
  source 1..109: note = Optionally with the N-terminus acetylated and/or the C-terminus amidated
EDASSDSTGADPAQEAGTSKPNEEISGPAEPASPPETTTTAQETSAAAVQGTAKVTSSRQELNPLKSIVEKSILLTEQALAKAGKGMHGGVPGGKQFIENGSEFAQKLL

SEQ ID NO: 8 - protein, 104 aa, Homo sapiens
  source 1..104: note = Optionally with the N-terminus acetylated and/or the C-terminus amidated
EDASSDSTGADPAQEAGTSKPNEEISGPAEPASPPETTTTAQETSAAAVQGTAKVTSSRQELNPLKSIVEKSILLTEQALAKAGKGMHGGVPGGKQFIENGSEF

SEQ ID NO: 9 - protein, 14 aa, Homo sapiens
  source 1..14: note = Optionally with the N-terminus acetylated and/or the C-terminus amidated
NGSEFAQKLLKKFS